(19) United States
(12) Patent Application Publication
(10) Pub. No.: US 2026/0081038 A1
(43) Pub. Date: Mar. 19, 2026

(54) DEVICES, SYSTEMS, AND METHODS FOR OPTIMIZING PATIENT RECOVERY

(71) Applicant: Howmedica Osteonics, Corp., Mahwah, NJ (US)

(72) Inventors: Vivienne CLAYTON, Montville, NJ (US); Emily HAMPP, Far Hills, NJ (US); Jillian TULIEBITZ, London (GB); Pankaj JOSHI, New Delhi (IN)

(73) Assignee: Howmedica Osteonics Corp, Mahwah, NJ (US)

(21) Appl. No.: 19/330,904

(22) Filed: Sep. 17, 2025

Related U.S. Application Data
(60) Provisional application No. 63/696,193, filed on Sep. 18, 2024.

(51) Int. Cl. G16H 50/70; G16H 10/60; G16H 20/00; G16H 40/00; G16H 50/20; G16H 80/00

(52) U.S. Cl. CPC G16H 50/70; G16H 10/60; G16H 20/00; G16H 40/00; G16H 50/20; G16H 80/00

(57) ABSTRACT

A method may include receiving at least one of preoperative activity levels or postoperative activity levels associated with an instant patient from a patient database, receiving a patient preference associated with the instant patient from the patient database, determining, based on at least one of the preoperative activity levels or the postoperative activity, one or more potential connections from a plurality of users, and determining a time interval. The time interval may be based on a date of surgery for the instant patient and a present date. The method may further include determining a search area. The search area may be based on a current location of a user equipment associated with the instant patient. The method may further include determining one or more correlated users, based on (i) at least one of the preoperative activity levels or the postoperative activity levels and (ii) the one or more potential connections.

### CROSS-REFERENCE TO RELATED APPLICATION(S)

This patent application is a continuation of and claims the benefit of priority to U.S. Provisional Application No. 63/696,193, filed on Sep. 18, 2024, the entirety of which is incorporated herein by reference.

### FIELD OF THE DISCLOSURE

The present disclosure relates to systems and methods for optimizing patient outcomes, and in particular to a system and a method for determining preoperative and postoperative activities to optimize outcomes after joint replacement procedures, among other aspects.

### BACKGROUND OF THE DISCLOSURE

Social interaction may be beneficial to patients recovering from surgeries, such as total knee arthroplasty (TKA). Patients with greater levels socialization and physical activity may correspond with improved patient outcomes. A variety of data sources may be used to analyze a patient and connect them with one or more similar patients, and to make relevant suggestions to the patient. Improved computer and algorithmic systems and methods for performing, collecting, and analyzing data to assist in patient recovery are desired.

### BRIEF SUMMARY OF THE DISCLOSURE

Aspects of the disclosure relate to, among other things, systems, devices, and methods for providing relevant clinical connections and quantitative pain evaluation, among other aspects. Each of the aspects disclosed herein may include one or more of the features described in connection with any of the other disclosed aspects.

According to an example, a method for providing relevant clinical connections may include receiving at least one of preoperative activity levels or postoperative activity levels associated with an instant patient from a patient database, receiving a patient preference associated with the instant patient from the patient database, determining, based on at least one of the preoperative activity levels or the postoperative activity, one or more potential connections from a plurality of users, and determining a time interval. The time interval may be based on a date of surgery for the instant patient and a present date. The method may further include determining a search area. The search area may be based on a current location of a user equipment associated with the instant patient. The method may further include determining one or more correlated users, based on (i) at least one of the preoperative activity levels or the postoperative activity levels and (ii) the one or more potential connections and displaying the one or more correlated users on an electronic display.

Any of the systems, devices, and methods described herein may include any of the following features. The patient preference may include a travel preference and a connection preference. The determining the search area may be further based on the travel preference. The preoperative activity levels and the postoperative activity levels may include kinematics data. The kinematics data may be generated by a wearable sensor coupled to the instant patient, or a smart implant implanted in the instant patient. The kinematics data may include a range of motion, stiffness, or laxity of a joint. The smart implant may be installed at the joint. The connection preference may include at least one of an activity partner, a recovery mentor, or a recovery mentee. The one or more potential connections may be further based on a plurality of connections preferences, each of the plurality of connection preferences associated with a user of the plurality of users. Each of the one or more potential connections may have at least one characteristic in common with the instant patient. The one or more correlated users may be displayed in descending order of most correlated with the instant patient to least correlated with the instant patient. The determining the one or more potential connections from the plurality of users may be further based on pain data of the instant patient. The pain data of the instant patient may be based on at least one of a facial expression classifier, a movement classifier, or a real-time sensor information classifier. The facial expression classifier may be based on receiving visual media of the instant patient, the visual media may include the instant patient performing at least one movement. The method may further include determining one or more parameters from the visual media using facial detection, facial alignment, and/or facial normalization, determining at least one facial landmark or a facial texture from the determined one or more parameters, and evaluating a facial pain instance based on the determined at least one facial landmark or facial texture. The at least one facial landmark may include eyes of the instant patient. The pain data may be further based on a movement classifier. The movement classifier may be based on kinematics data generated by a wearable sensor coupled to the instant patient, or a smart implant implanted in the instant patient. The real-time sensor information classifier may be based on at least one real-time measurement by a smart implant implanted in the instant patient. The preoperative activity levels and the postoperative activity levels may further include range of motion data, alignment data, or joint stiffness data.

According to another example, a method for providing relevant clinical suggestions may include receiving at least one of preoperative activity levels or postoperative activity levels associated with an instant patient from a patient database, receiving pain data from a patient database, the pain data associated with the instant patient, determining a time interval. The time interval may be based on a date of surgery for the instant patient and a present date. The method may further include determining, based on (i) the preoperative activity levels or the postoperative activity levels, (ii) the pain data, and (iii) the time interval, a clinical suggestion for the patient. The clinical suggestion may include at least one physical activity.

Any of the systems, devices, and methods described herein may include any of the following features. The pain data may be based on a facial expression classifier, a movement classifier, and a real-time sensor information classifier. The facial expression classifier may be based on receiving visual media of the instant patient. The visual media may include the instant patient performing at least one movement. The method may further include determining at least one facial landmark or a facial texture using facial detection, alignment and/or normalization of the visual media and evaluating a facial pain instance based on the determined at least one facial landmark or facial texture.

According to another example, a method for evaluating pain may include determining a facial expression classifier. Determining the facial expression classifier may include receiving visual media of an instant patient from a patient database. The visual media may include the instant patient performing at least one movement. The determining the facial expression classifier may include determining at least one facial landmark or a facial texture using facial detection, alignment and/or normalization of the visual media and evaluating a facial pain instance based on the determining at least one facial landmark or facial texture. The method may further include determining a movement classifier based on kinematics data generated by a wearable sensor coupled to the instant patient, or a smart implant implanted in the instant patient, determining a real-time sensor information classifier based on at least one real-time measurement by a smart implant implanted in the instant patient, and displaying a pain metric associated with the instant patient, based on the facial expression classifier, the movement classifier, and the real-time sensor information classifier.

Any of the systems, devices, and methods described herein may include any of the following features. The method may further include comparing the preoperative pain metric to a postoperative pain metric and generating a clinical suggestion, based on the comparing the preoperative pain metric to the postoperative pain metric.

### BRIEF DESCRIPTION OF THE DRAWINGS

A more complete appreciation of the subject matter of this disclosure and the various advantages thereof may be realized by reference to the following detailed description, in which reference is made to the following accompanying drawings:

FIG. 1 is a flow chart illustrating a system for collection, transmission, and storage of preoperative, intraoperative, and postoperative data, and outputs of the system according to aspects of this disclosure.

FIG. 2 is a schematic diagram depicting the processing of preoperative, intraoperative, and postoperative data and outputs of the system of FIG. 1, according to aspects of this disclosure.

FIG. 3 is a schematic diagram exemplifying types of preoperative and intraoperative data and outputs of the system of FIG. 1, according to aspects of this disclosure.

FIG. 4 is a schematic diagram exemplifying types of postoperative data and outputs of the system of FIG. 1, according to aspects of this disclosure.

FIGS. 5 and 6 show exemplary legs and bones of a leg, respectively, and showing various mechanical axes which may be measured as part of kinematic data included in FIGS. 3-4, according to aspects of this disclosure.

FIG. 7 illustrates a preoperative measurement system configured to collect preoperative data, according to aspects of this disclosure.

FIGS. 8-9 illustrate exemplary wearable sensors, according to aspects of this disclosure.

FIG. 10 illustrates an intraoperative measurement system configured to collect intraoperative data, according to aspects of this disclosure.

FIGS. 11-13 illustrate exemplary sensored medical devices, according to aspects of this disclosure.

FIG. 14 illustrates a postoperative measurement system configured to collect postoperative data, according to aspects of this disclosure.

FIGS. 15-16 illustrate an exemplary sensored patient bed, according to aspects of this disclosure.

FIG. 17 is a flow chart illustrating exemplary preoperative, intraoperative, and postoperative algorithms for the system of FIG. 1, according to aspects of this disclosure.

FIG. 18 is a flow chart illustrating a first exemplary method for facial pain evaluation, according to aspects of this disclosure.

FIG. 19 is a flow chart illustrating a second exemplary method for facial pain evaluation, according to aspects of this disclosure.

FIG. 20 is a flow chart illustrating a method for generating a quantitative pain metric, according to aspects of this disclosure.

FIG. 21 illustrates an exemplary user interface for user preferences, according to aspects of this disclosure.

FIG. 22 is a flow chart illustrating a method for generating a clinically relevant suggestions, according to aspects of this disclosure.

FIGS. 23A-23B illustrate an exemplary user interface for clinically relevant suggestions, according to aspects of this disclosure.

FIG. 24 is a flow chart illustrating a method for generating patient connections, according to aspects of this disclosure.

FIG. 25 illustrates an exemplary user interface for a patient recovery application, according to aspects of this disclosure.

FIG. 26A illustrates an exemplary user interface for patient connections, according to aspects of this disclosure.

FIG. 26B illustrates an exemplary user interface for patient meetups, according to aspects of this disclosure.

FIG. 27 illustrates an exemplary user interface for a non-patient partner, according to aspects of this disclosure.

### DETAILED DESCRIPTION

Reference will now be made in detail to the various embodiments of the present disclosure illustrated in the accompanying drawings. Wherever possible, the same or like reference numbers will be used throughout the drawings to refer to the same or like features. It should be noted that the drawings are in simplified form and are not drawn to precise scale. Additionally, the term “a,” as used in the specification, means “at least one.” The terminology includes the words above specifically mentioned, derivatives thereof, and words of similar import. Although at least two variations are described herein, other variations may include aspects described herein combined in any suitable manner having combinations of all or some of the aspects described.

As used herein, the terms “implant trial” and “trial” will be used interchangeably and as such, unless otherwise stated, the explicit use of either term is inclusive of the other term. In this disclosure, “user” is synonymous with “practitioner” and may be any person completing the described action (e.g., surgeon, technician, nurse, etc.).

An implant may be a device that is at least partially implanted in a patient and/or provided inside of a patient's body. For example, an implant may be a sensor, artificial bone, or other medical device coupled to, implanted in, or at least partially implanted in a bone, skin, tissue, organs, etc. A prosthesis or prosthetic may be a device configured to assist or replace a limb, bone, skin, tissue, etc. Many prostheses are implants, such as a tibial prosthetic component. Some prostheses may be exposed to an exterior of the body and/or may be partially implanted, such as an artificial forearm or leg. Some prostheses may not be considered implants and/or otherwise may be fully exterior to the body, such as a knee brace. Systems and methods disclosed herein may be used in connection with implants, prostheses that are implants, and also prostheses that may not be considered to be “implants” in a strict sense. Therefore, the terms “implant” and “prosthesis” will be used interchangeably and as such, unless otherwise stated, the explicit use of either term is inclusive of the other term. Although the term “implant” is used throughout the disclosure, this term should be inclusive of prostheses which may not necessarily be “implants” in a strict sense.

In describing preferred embodiments of the disclosure, reference will be made to directional nomenclature used in describing the human body. It is noted that this nomenclature is used only for convenience and that it is not intended to be limiting with respect to the scope of the invention. For example, as used herein, the term “distal” means toward the human body and/or away from the operator, and the term “proximal” means away from the human body and/or towards the operator. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such system, process, method, article, or apparatus. The term “exemplary” is used in the sense of “example,” rather than “ideal.” Further, relative terms such as, for example, “about,” “substantially,” “approximately,” etc., are used to indicate a possible variation of ±10% in a stated numeric value or range.

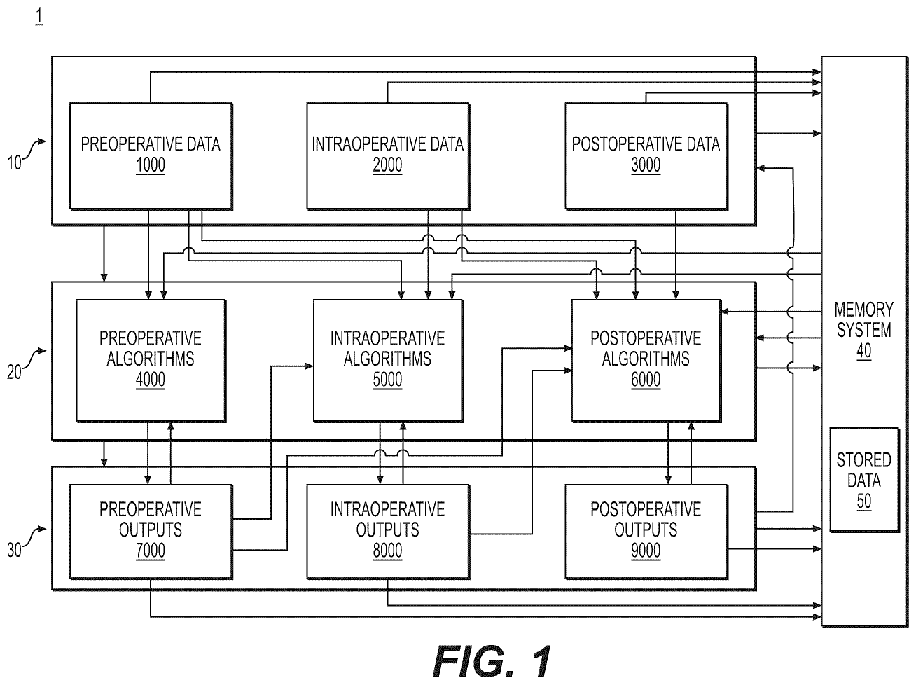

FIG. 1 illustrates an electronic data processing system 1 for collecting, storing, processing, and outputting data throughout the course of treatment of a patient.

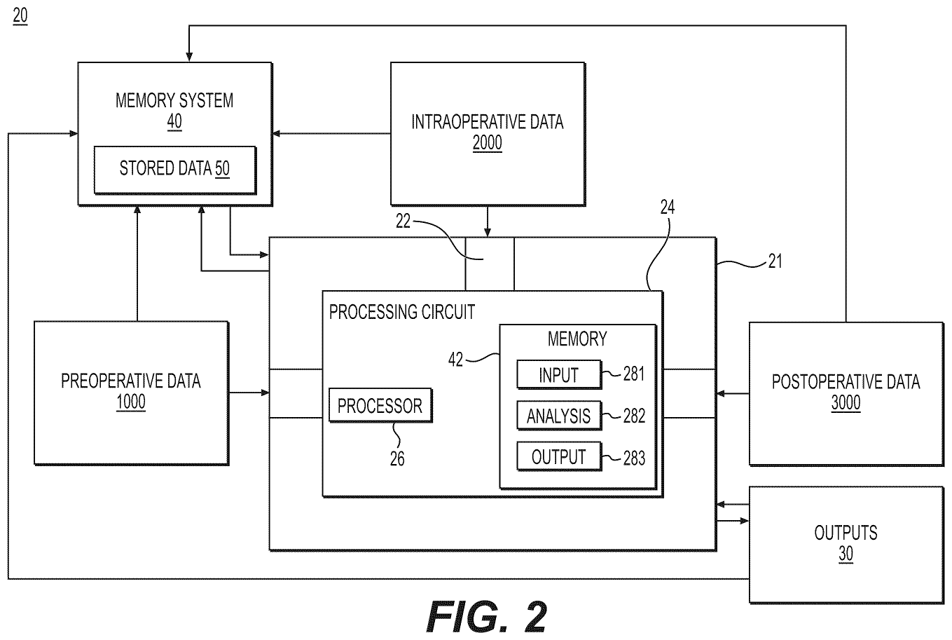

Referring to FIG. 1, input information 10 may be input into a system or module 20 to generate output information 30, which may be fed back into system 20 as input information 10. System 20 may be an artificial intelligence (AI) and/or machine learning system. System 20 may include an AI module 21 (shown in FIG. 2), which may include or communicate with a memory system 40 configured to store the plurality of inputs or input information 10, outputs or output information 30, and stored data 50 from prior patients and/or prior procedures. Input information 10 and output information 30 of an instant procedure may also become stored data 50 and/or used as input information 10 into system 20 and/or memory system 40. Although certain information is described in this specification as being input information 10 or output information 30, due to the continuous feedback loops of data (which may be anchored by memory system 40), input information 10 described herein may alternatively be determinations or output information 30, and output information 30 described herein may also be used as input information 10. For example, some input information 10 may be directly sensed or otherwise received, and other input information 10 may be determined or output based on other input information 10.

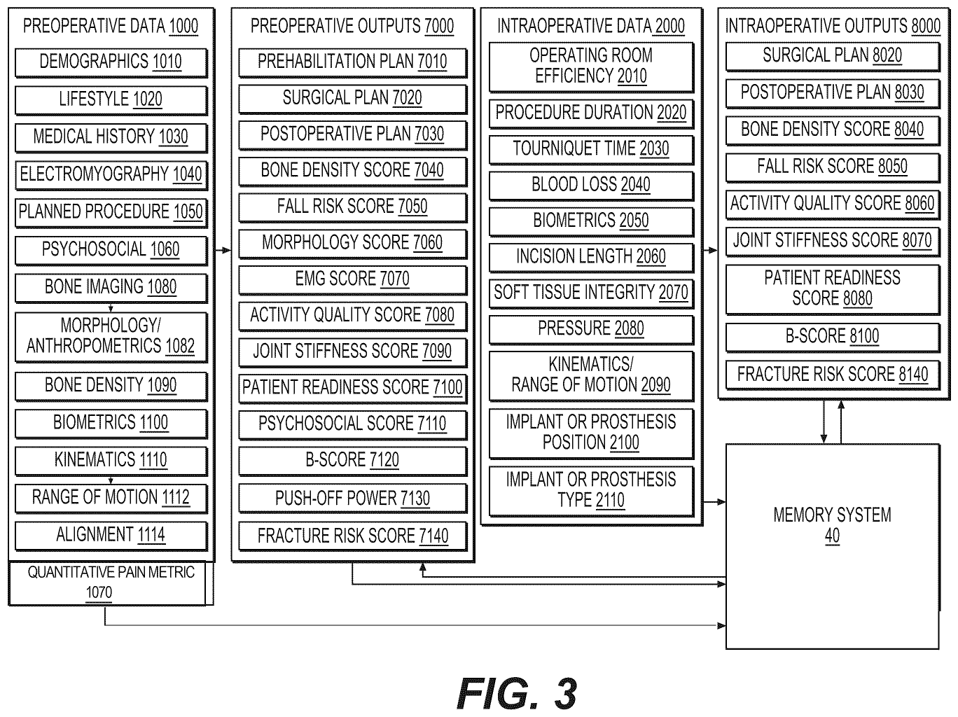

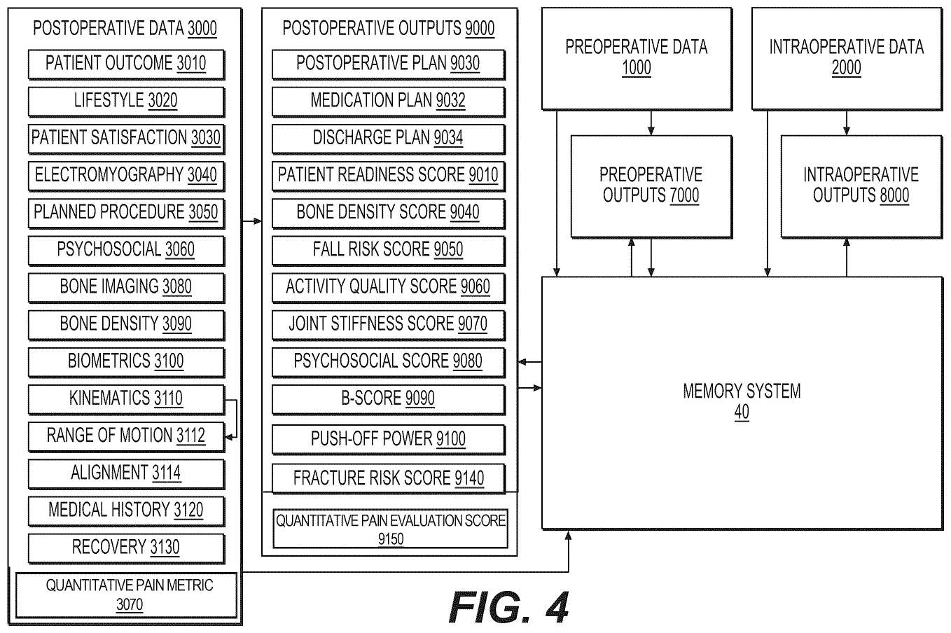

Input information 10 may include preoperative data 1000, intraoperative data 2000, and postoperative data 3000. System 20 may perform a plurality of algorithms, such as preoperative algorithms 4000, intraoperative algorithms 5000, and postoperative algorithms 6000 to generate output information 30. Output information 30 may include preoperative outputs 7000, intraoperative outputs 8000, and postoperative outputs 9000. Some or all of preoperative outputs 7000, intraoperative outputs 8000, and postoperative outputs 9000 may include determinations such as guidance for medical procedures, guidance for preoperative or pre-habilitation treatment plans, guidance for postoperative or recovery plans, etc., as will be described in more detail hereinafter. System 20 may include one or more algorithms or modules configured to aggregate results from multiple preoperative algorithms 4000, intraoperative algorithms 5000, and/or postoperative algorithms 6000 to compile algorithm determinations for certain outputs (e.g., surgical plans, medical treatment plans, or instructions). As shown by the arrows in FIG. 1, preoperative outputs 7000, intraoperative outputs 8000, and postoperative outputs 9000 may become inputs into system 20 and/or memory system 40. Details of input information 10 and output information 30 will be described with reference to FIGS. 4-3.

Preoperative data 1000 may be data collected, received, and/or stored prior to an initiation of a medical treatment plan or medical procedure. Intraoperative data 2000 may be data collected, received, and/or stored during a medical treatment plan or medical procedure. Although the term “intraoperative” is used, the word “operative” should not be interpreted as requiring a surgical operation. Postoperative data 3000 may be collected, received, and/or stored after completion of the medical treatment or medical procedure.

FIG. 2 illustrates an exemplary system architecture for system 20. Referring to FIG. 2, AI module 21 may be implemented using one or more computing platforms. Examples of one or more computing platforms may include, but are not limited to, smartphones, wearable devices, tablets, laptop computers, augmented, virtual, extended, and/or mixed reality systems, desktop computers, Internet of Things (IoT) device, remote server/cloud based computing devices, user equipment, or other mobile or stationary devices. AI module 21 may also include one or more hosts or servers connected to a networked environment through wireless or wired connections. Remote platforms may be implemented in or function as base stations (which may also be referred to as Node Bs, evolvedNodeBs (eNBs), gNodeBs). Remote platforms may also include web servers, mail servers, application servers, large-language model (LLM) servers, etc.

AI module 21 may include at least one communication module or interface 22 and a processing circuit 24. Processing circuit 24 may include one or more processors 26 and a memory or storage 42. Memory or storage 42 may be a part of memory system 40. Memory system 40 is shown in FIG. 2 as providing separate storage from AI module 21 to exemplify that large amounts of data (e.g., stored data 50) may be stored separately and sent to AI module 21 via communication module 22 when needed or where appropriate. However, memory system 40 may be a part of a computing platform for AI module 21.

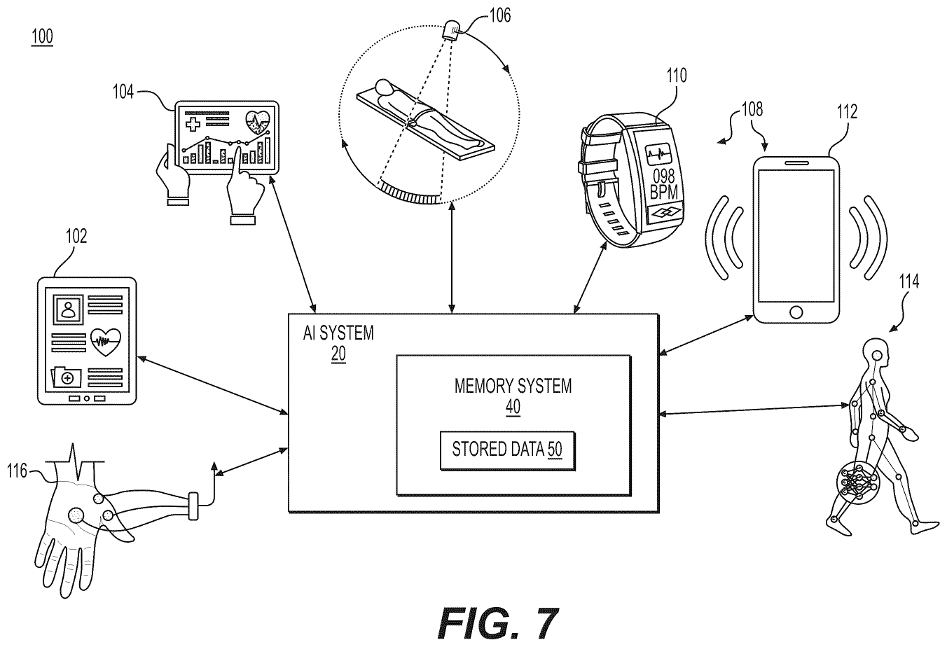

AI module 21 may be configured to receive plurality of inputs 10 (e.g., preoperative data 1000, intraoperative data 2000, and postoperative data 3000), and/or stored data 50 from prior procedures or patients, via communication module 22. Preoperative data 1000, intraoperative data 2000, and postoperative data 3000 may be received via manual input or from the various sensors discussed with references to FIGS. 7-13. Plurality of inputs 10 may be stored in memory 42 and/or memory system 40. Plurality of input information 10 may be analyzed by processor 24 to determine patterns. AI module 21 may be configured to perform preoperative algorithms 4000, intraoperative algorithms 5000, and postoperative algorithms 6000 via processing circuit 24, and to generate output information 30 via processor 26.

Communication module 22 may enable wireless communications between system 20 and the various sensors or data collection devices described herein. Communication module 22 may include wired or wireless interfaces (e.g., jacks, antennas, transmitters, receivers, transceivers, wire terminals, etc.) for conducting data communications with external sources via a direct connection or a network connection (e.g., an Internet connection, a LAN, WAN, or WLAN connection, LTE, 4G, 5G, Bluetooth, near field communication (NFC), radio frequency identifier (RFID), ultra-wideband (UWB), future contemplated networking standards such as 6G, etc.). Communication module 22 may include a radio interface including filters, converters (for example, digital-to-analog converters and the like), mappers, a Fast Fourier Transform (FFT) module, and the like, to generate symbols for a transmission via one or more downlinks and to receive symbols (for example, via an uplink). Communication module 22 may include a Bluetooth module, Wi-Fi module, etc. to receive the input information 10. For example, communication module 22 may include an Ethernet card and port for sending and receiving data via an Ethernet-based communications link or network. In another example, communication module 22 may include a Wi-Fi transceiver for communication via a wireless communications network.

Processing circuit 24 may be configured to implement various functions (e.g., calculations, processes, analyses) described herein. Processor 26 may be implemented as a general purpose processor or computer, special purpose computer or processor, microprocessor, digital signal processor (DSPs), an application specific integrated circuit (ASIC), one or more field programmable gate arrays (FPGAs), a group of processing components, processor based on a multi-core processor architecture, or other suitable electronic processing components. Processor 26 may be configured to perform machine readable instructions, which may include one or more modules implemented as one or more functional logic, hardware logic, electronic circuitry, software modules, etc. In some cases, processor 26 may be remote from one or more of the computing platforms comprising module 21 and/or system 20. Processor 26 may be configured to perform one or more functions associated with AI module 21, such as precoding of antenna gain/phase parameters, encoding and decoding of individual bits forming a communication message, formatting of information, and overall control of one or more computing platforms comprising AI module 21, including processes related to management of communication resources and/or communication module 22.

Memory 42 may provide an example of the types of devices comprising the memory system 40. Memory 42 may be one or more external or internal devices (random access memory or RAM, read-only memory or ROM, flash-memory, hard disk storage or HDD, solid state devices or SSD, static storage such as a magnetic or optical disk, other types of non-transitory machine or computer readable media, etc.) configured to store data and/or computer readable code and/or instructions that completes, executes, or facilitates various processes or instructions described herein. Memory 42 may be or include volatile memory or non-volatile memory (e.g., semiconductor-based memory device, a magnetic memory device and system, an optical memory device and system, fixed memory, or removable memory). Memory 42 may include database components, object code components, script components, or any other type of information structure to support the various activities described herein. In some aspects or embodiments, memory 42 may be communicably connected to processor 26 and may include computer code to execute one or more processes described herein. Memory 42 may contain a variety of modules, each capable of storing data and/or computer code related to specific types of functions. In some embodiments, memory 42 may contain several modules related to medical procedures, such as an input module 281, an analysis module 282, and an output module 283. Input module 281 may receive input information 10, and output module 283 may output (e.g., display or transmit) output information 30. Analysis module 282 may include and/or operate preoperative algorithms 4000, intraoperative algorithms 5000, and postoperative algorithms 6000.

AI module 21 and/or system 20 need not be contained in a single housing. Rather, components of AI module 21 may be located in various different locations or in a remote location. Components of module 21, including components of processor 26 and memory 42, may be located, for example, in components of different computers, robotic systems, devices, etc. used in surgical procedures.

FIGS. 3-4 illustrate the types of input information 10 and output information 30, and FIGS. 7-14 illustrate examples of various output information 30 and how various other input information 10 may be measured. Preoperative data 1000, intraoperative data 2000, and postoperative data 3000 may be collected using preoperative, intraoperative, and postoperative measurement systems 100, 200, and 300. Preoperative algorithms 4000, intraoperative algorithms 5000, and postoperative algorithms 6000 may be used to generate preoperative outputs 7000, intraoperative outputs 8000, and postoperative outputs 9000.

### Preoperative Data 1000

Preoperative data 1000 may include any information collected by memory system 40 prior to a medical procedure, such as a surgical procedure or other patient treatment event. Referring to FIGS. 3-4, preoperative data 1000 may include information on demographics 1010, lifestyle 1020, medical history 1030, electromyography (EMG) 1040, planned procedure 1050, psychosocial information 1060 (including a quantitative pain metric 1070), bone imaging 1080, bone density 1090, biometrics 1100, and kinematics 1110. This list, however, is not exhaustive and preoperative data 1000 may include other patient specific information. Some of preoperative data 1000 may be directly sensed via one or more devices, may be manually entered by a medical professional, patient, or other party, and other preoperative data 1000 may be determined (e.g., using a preoperative algorithm 4000) based on directly sensed information, input information, and/or stored information from prior medical procedures.

Demographics 1010 may include patient age, gender, height, weight, nationality, ethnicity/race, education, income, marital status, occupation, body mass index (BMI), etc. Lifestyle 1020 may include information on smoking habits, drug use (including drug addiction), exercise habits, drinking habits, eating habits, fitness, thrill-seeking habits and/or risk adverse traits, a type of vehicle a patient drives and movements associated with entering and exiting the vehicle, a type of house or residence the patient lives in and movements associated with climbing and descending stairs, bending movements during daily activities, etc. In some examples, demographics 1010 may include the average amount of lateral movement and/or average amount of ascending or descending movement (e.g. climbing stairs or walking up a hill, etc.).

Medical history 1030 may include allergies, disease progressions, addictions, prior medication use, prior drug use, prior infections, prior immunizations, prior illnesses, disabilities, and/or diseases, comorbidities, prior surgeries or treatment, prior injuries, prior pregnancies, utilization of orthotics, braces, prosthetics, or other medical devices, etc. EMG data 1040 may include information on a muscle response or electrical activity in response to a nerve's stimulation.

Information on a planned procedure 1050 may include information about a planned site of the procedure, a disease or infection state, type of procedure to be performed, etc. Alternatively, or in addition thereto, a planned procedure 1050 may include a surgeon's surgical or other procedure or treatment plan (planned steps or instructions on incisions, bone cuts, implant design, implant alignment, etc.) that was manually prepared by a surgeon and/or previously prepared using one or more algorithms. Psychosocial information 1060 may include perceived pain, stress level, anxiety level, mental health status, other feelings and psychosocial data, and other patient reported outcome measures (PROMS). Psychosocial information 1060 may include mental health status and/or information from a Veteran's Rand-12 (VR-12) mental component summary (MCS).

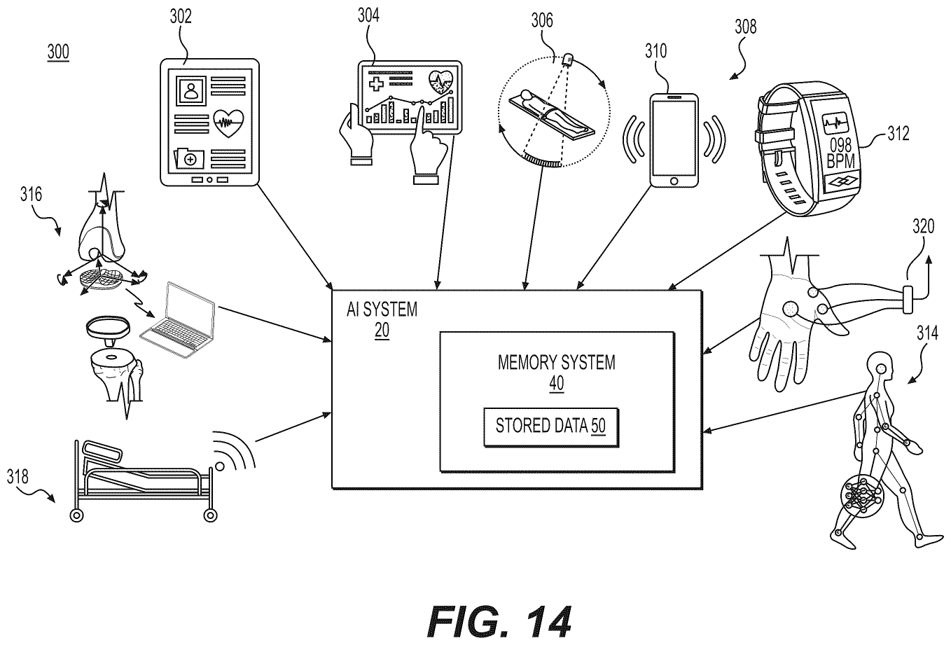

Psychosocial information 1060 may include a quantitative pain metric (QPM) 1070. QPM 1070 may be derived from one or more data sources, including a facial classifier 1070(a) using facial expressions recorded by a camera (such as a mobile device camera), a movement classifier 1070(b) for evaluation of movement of a particular body part as recorded by the camera, and an implant movement classifier 1070(c). Further discussion of QPM 1070 is provided with respect to FIGS. 14-17.

Bone imaging data 1080 may include morphology and/or anthropometrics 1082 (e.g., physical dimensions of internal organs, bones, etc.), fractures, slope or angular data, tibial slope, posterior tibial slope or PTS, bone density 1090 (e.g., bone mineral or bone marrow density, bone softness or hardness, or bone impact), etc. Bone density 1090 may be collected separately from bone imaging information 1080 and/or may be collected, for example, using indent tests or a microindentation tool. Bone imaging data 1080 may not be limited to strictly “bone” and may be inclusive of other internal imaging data, such as cartilage, soft tissue, or ligaments.

Bone imaging data 1080 may include or be used to determine alignment data 1114. Bone imaging data 1080, alignment data 1114, and/or morphology and/or anthropometrics 1082 may include data on bone landmarks (e.g., condyle surface, head or epiphysis, neck or metaphysis, body or diaphysis, articular surface, epiconcyle, process, protuberance, tubercle vs tuberosity, trochanter, spine, linea or line, facet, crests and ridges, foramen and fissure, meatus, fossa and fovea, incisure and sulcus, and sinus) and/or bone geometry (e.g., diameters, slopes, angles) and other anatomical geometry data. Such geometry is not limited to overall geometry and may include specific lengths or thicknesses (e.g., lengths or thicknesses of a tibia or femur). Bone imaging data 1080, alignment data 1114, and/or morphology and/or anthropometrics 1082 may also include data on soft tissues for ligament insertions and/or be used to determine ligament insertion sites. For example, bone density 1090 may be determined from bone imaging data 1080 and may be used to locate or determine a ligament insertion site to balance a knee.

Bone imaging data 1080, alignment data 1114, and/or morphology and/or anthropometrics 1082 may include lower extremity mechanical alignment, lower extremity anatomical alignment, femoral articular surface angle, tibial articular surface angle, mechanical axis alignment strategy, anatomical alignment strategy, natural knee alignment strategy, femoral bowing, tibial bowing, patello-femoral alignment, coronal plane deformity, coronal plane deformity that can be passively correctable, sagittal plane deformity, extension motion, flexion motion, anterior cruciate ligament (ACL) ligament intact, posterior cruciate ligament (PCL) ligament intact, knee motion in all three planes during active and passive range of motion in a joint, three dimensional size, proportions and relationships of joint anatomy in both static and motion, height of a joint line, lateral epicondyle, medial epicondyle, lateral femoral metaphysical flare, medial femoral metaphyseal flare, proximal tibio-fibular joint, tibial tubercle, coronal tibial diameter, femoral interepicondylar diameter, femoral intermetaphyseal diameter, sagittal tibial diameter, posterior femoral condylar offset-medial and lateral, lateral epicondyle to joint line distance, and/or tibial tubercle to joint line distance.

Biometrics 1100 may include resting heart rate or heat rate variability, electrocardiogram data, breathing rate, temperature (e.g., internal or skin temperature), skin moisture, oxygenation, sleep patterns (e.g., heart rate variability or HRV, REM cycle data, type of sleep such as REM, deep, or light, sleep frequency, time asleep versus time awake, disturbances in the sleep or periods of movement, patterns in sleep timing or time of day asleep, etc.), and/or activity frequency, type, and intensity. Biometrics 1100 may include patient-specific or unique characteristics, such as fingerprint data, DNA or RNA signatures, etc.

Kinematics 1110 may include movement or position information at various anatomical areas or locations, muscle function or capability, and range of motion 1112 data. Additional kinematics 1110 data may include strength measurements and/or force measurements. For example, kinematics 1110 may include data used to determine a push-off power, force, or acceleration, or a power, force, or acceleration at a toe during walking. Range of motion 1112 data may include a range of motion at one or more joints, such as an angular range or axes of joint motion, or flexion or extension data. For example, kinematics 1110 may include a flexion value, where a flexion value of 180 degrees±3 degrees may indicate a full extension of a joint, and any value other than 180 degrees±3 degrees may indicate a joint in flexion where bones on either side of the joint intersect to form an angle other than 180 degrees. Kinematics 1110 may include dynamic information, speed, or acceleration information, torque, or force information, etc. Some of this information may be estimated or determined based on raw data from motion sensor systems 114 and/or other sensors. For example, kinematics 1110 may include how quickly a patient can bend a joint, sit down, stand up, a push-off power during walking, etc. Kinematics 1110 may also include steps (e.g., measured by a pedometer) and/or measured gait. Kinematics 1110 may include a number of fall events and/or disoriented events (e.g., measured by an accelerometer, mobile device 108, etc.)

Kinematics 1110 may include swaying or other movement which would indicate an unsteady balance of a patient, such as postural swaying at the hips, knees, or neck. Kinematics 1110 may include pendulum knee drop information. Kinematics 1110 may also include and/or indicate frailty, fall risk, and/or joint stiffness (e.g., based on a speed or case of how a joint is moved through a range of motion).

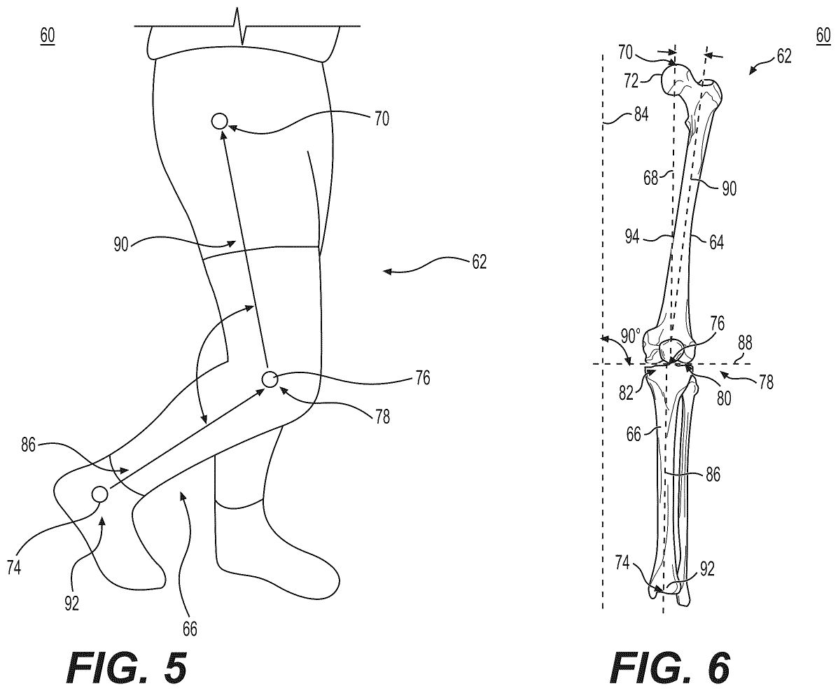

Kinematics information 1110 may include measurements in relation to a leg axis system 60 (FIGS. 5-6), such as alignment data 1114 or other anatomical measures. Alignment data 1114 may be obtained using kinematics information 1110 and/or range of motion information 1112, bone imaging data 1080 and/or morphology/anthropometrics data 1082, etc. In this way, alignment data 1114 may also be a type of preoperative output 7000. Anatomical measures and/or alignment data 1114 may include arithmetic hip-knee-ankle angle or aHKA, anatomical hip-knee-ankle angle, medial proximal tibial angle or MPTA, lateral distal femoral angle or LDFA, mechanical axis alignment, anatomic alignment, natural knee alignment, gap balancing, measured resection, etc., and these values may be combined. For instance, a joint line may be a sum of MPTA and LDFA, and a hip-knee-ankle angle or HKA may be a difference between MPTA and LDFA. These values may be used as coordinates on a 2D plane to describe a patient's knee anatomy.

Preoperative data 1000 and/or information stored in memory system 40 may also include known data and/or data from third parties, such as data from the Knee Society Clinical Rating System (KSS) or data from the Western Ontario and McMaster Universities Osteoarthritis Index (WOMAC).

FIGS. 5-6 illustrate leg axis system 60. However, aspects disclosed herein are not limited to enhancing alignment of a leg or a knee joint and may enhance alignment and/or functions at other joints or body parts. Referring to FIGS. 5-6, leg axis system 60 may be relative to a leg 62. Leg 62 may be a right or left leg. Leg 62 may comprise a femur 64 and a tibia 66. A mechanical axis 68 of leg 62 may be illustrated by a dashed line drawn through a center 70 of a femoral head 72 (at a hip joint) to a center 74 of ankle joint 96. Mechanical axis 68 may extend through a center 76 of a knee joint 78 at approximately a medial tibial spine 94. Knee joint 78 may comprise lateral articular surfaces and/or compartments 80 and medial articular surfaces and/or compartments 82 that support leg movement. Alignment of leg 62 to mechanical axis 68 may minimize wear on articular surfaces of a prosthetic knee joint and may reduce mechanical stress on wear surfaces of femur 64 and tibia 66. Similarly, alignment to mechanical axis 68 of leg 62 may reduce stress on any prosthetic components coupled to femur 64 and/or tibia 66. Alignment of knee joint 78 may further include balancing between lateral compartment 80 and medial compartment 82 of knee joint 78.

A vertical axis 84 is shown by a dashed line drawn relative to mechanical axis 68 and an anatomical axis 86 of tibia 66. A horizontal axis 88 is shown by a dashed line that is perpendicular to vertical axis 84. Horizontal axis 88 is shown extending through center 76 of knee joint 78 between a distal end of femur 64 and a proximal end of tibia 66. Vertical axis 84 may align with the pubic symphysis, which is a midline cartilaginous joint in proximity to a pelvic region. An anatomical axis 90 of femur 64 is illustrated by dashed line 90. The anatomical axis 90 of femur 64 may traverse an intramedullary canal of femur 64. The anatomical axis 86 of tibia 66 may traverse an intramedullary canal of tibia 66. Mechanical axis 68 and anatomical axis 86 of tibia 66 may lie along a same line or be the same from knee joint 78 to center 74 of ankle joint 96 of leg 62.

Femur 64 and tibia 66 can be misaligned to mechanical axis 68 of leg 62. In an aligned leg, mechanical axis 68 may form an angle of approximately 3 degrees with the vertical axis when the leg is fully extended. A surgeon may install prosthetic components in a knee joint 78 aligned to mechanical axis 68 of leg 62 to optimize reliability and performance of knee joint 78. An alignment process may include measurement of leg misalignment (e.g., the offset of anatomical axis 90 from mechanical axis 68) and determination of the required compensation to align leg 62 to mechanical axis 68 within a predetermined range. The predetermined range may be determined by a prosthetic component manufacturer, or a medical practitioner based on clinical evidence that supports reliability and performance of knee joint 78 when misalignment is kept within the predetermined range.

### Preoperative Measurement System 100

Referring to FIGS. 2 and 7, system 20 may collect preoperative data 1000 from preoperative measurement or sensing system 100. Preoperative measurement system 100 may include electronic devices storing electronic medical records (EMR) 102, patient/user interfaces or applications 104 such as tablets, computers, and smartphones 112, diagnostic imaging systems 106, mobile devices 108, a motion sensor, pressure sensor, and/or kinesthetic sensing systems 114 (see paragraph et seq.), and electromyography or EMG systems 116. The devices of the preoperative measurement system 100 may each include one or more communication modules (e.g., Wi-Fi modules, Bluetooth modules, etc.) configured to transmit preoperative data 1000 to memory system 40, system 20, to each other, etc. System 20 may use other types of stimulation systems (e.g., configured for a kinematic or EMG response) to collect preoperative data 1000.

System 20 may collect patient reported data, practitioner assessments, etc. using EMR 102. For example, EMR 102 may be used to collect data on demographics 1010, medical history 1030, biometrics 1100, and information about a planned procedure 1050. Patient and/or user interfaces 104 may be implemented on mobile applications and/or patient management websites or interfaces such as OrthologIQ®. Patient interfaces 104 may present questionnaires, surveys, or other prompts for patients to enter psychosocial information 1060 such as perceived or evaluated pain, stress level, anxiety level, feelings, and other patient reported outcome measures (PROMS). Practitioners may also report psychosocial information 1060 (e.g., qualitative assessments or evaluations) via patient interfaces 104 or other interfaces. Patients may also report lifestyle information 1020 via patient interfaces 104. These patient interfaces 104 may be executed on other devices disclosed herein (e.g., using mobile devices 108 or other computers).

System 20 may collect imaging information from diagnostic imaging systems 106, which may include computed tomography (CT) scans, magnetic resonance imaging (MRI), x-rays, radiography, ultrasound, thermography, tactile imaging, elastography, nuclear medicine functional imaging, positron emission tomography (PET), single-photon emission computer tomography (SPECT), etc. System 20 may use these diagnostic imaging systems 106 to collect bone imaging information 1080, including morphology and/or anthropometrics 1082 fractures, and bone density 1090 (e.g., bone mineral density or bone marrow density).

Mobile devices 108 may include wearable 110 (including smart watches, smart rings, e.g., Oura®, Galaxy Ring®, etc.), smartphones 112, tablets, augmented reality systems, virtual reality systems, mixed reality systems, augmented reality systems, and other devices known in the art. Mobile devices 108 may execute patient interfaces 104. In some examples, mobile devices 108 may include sensors and/or applications, which system 20 may use to collect biometrics 1100 and other types of patient specific data. For example, mobile devices 108 (e.g., FitBit®, Apple Watch®, Hexoskin®, iPhone®, etc.) may use cameras, light sensors, barometers, global positioning systems (GPS), accelerometers, temperature sensors (e.g., battery temperature sensors), and/or pressure sensors. In some examples, mobile devices 108 may measure heart rate, electrocardiogram data, breathing rate, temperature, oxygenation, sleep patterns, and activity types, frequencies, and intensities.

System 20 may use EMG systems 116 to collect EMG data 1040. EMG systems 116 may include one or more electrodes attached to skin or muscle to measure electrical activity and/or responses to nerve stimulation. System 20 may use EMG data 1040 to determine nerve damage or disease information. EMG data 1040 may include information on muscle activity of various body segments including knee, hip, ankle, tibialis anterior, foot, lower back, shoulder, wrist, elbow, forearm, neck, etc.

System 20 may use motion sensor and/or kinesthetic sensing systems 114, which may include motion capture (mocap) systems, external motion sensors, wearable sensors, and/or sensors machine vision (MV) technology. Motion sensor systems 114 may measure motion using an optical or light sensor, an accelerometer, a gyroscope, a magnetometer, a compass, barometer, a global positioning system (GPS), a pressure sensor, etc.

System 20 may use motion capture systems, which may include markerless motion capture systems and other motion sensors (e.g., wearable sensors) to collect kinematics and range of motion data. External motion sensors may include cameras, optical sensors, infrared sensors, ultrasonic sensors, etc. mounted, for example, in an operating room to monitor motion, heat, etc.

Wearable sensors 114 may include heart-rate monitors, some mobile devices 108 (e.g., wearable 110), and other sensor systems configured to be worn by a patient and track movement (e.g., travel movement and kinematics of anatomy, such as joint motion). Wearable sensors 114 may include accelerometers, GPS chips, acoustical ranging, magnetometer, inclinometers, hybrid sensors, MEMs devices, etc. Wearable sensors 114 may also include MotionSense sensors, ZipLine sensors, and/or pedometers. Wearable sensors 114 may monitor more than motion, such as pressure, temperature, sweat/perspiration, input related to stress, input related to air circulation, air purity or quality of an environment, etc. Wearable sensors 114 may include pressure insole sensors and/or sensored shoes configured to measure pressure, a pressure distribution, a center of pressure, etc. when a user steps. Such wearable sensors 114 may also measure acceleration or force as a user lifts a leg to take a step. Pressure data from pressure insole sensors or sensored shoes may be used to determine or evaluate balance, heel strike, and/or push-off forces, which may be used to determine or evaluate frailty, fall risk, compensatory gait, and overall function.

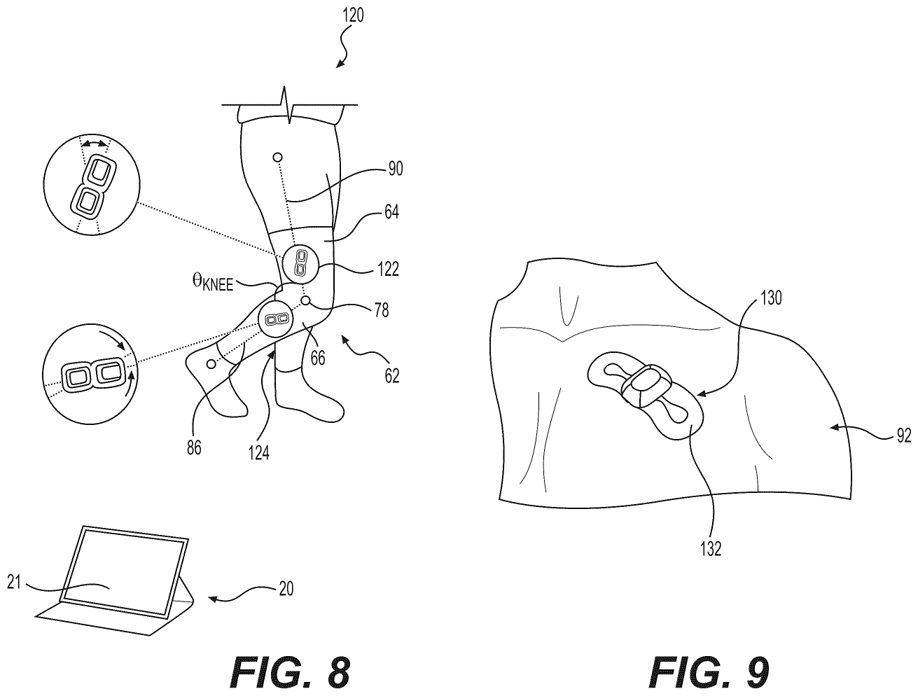

Referring to FIGS. 8-9, wearable sensors 114 may be implemented as a kinematics tracking system 120 and/or 130. As shown in the example of FIG. 8, kinematics tracking system 120 and/or 130 may be implemented as a tracking system 120 for a leg 62 (see also FIGS. 5-4). Tracking system 120 may include a first device 122 and a second device 124. System 20 and/or tracking system 120 may include a computer 21 having a display 28.

First device 122 may be coupled (e.g., adhered) to a first portion of a musculoskeletal system. Second device 124 may be coupled to a second portion of the musculoskeletal system. As shown in FIG. 8, first device 122 may be coupled to a thigh or femur 64 to move with femur 64. Second device 124 may be coupled to a calf or tibia 66 to move with tibia 66.

First and second devices 122 and 124 may be configured to measure a relative orientation between the first and second portions to determine an angle, such as an angle of knee joint 78 between the thigh and half. Since an orientation of first device 122 and/or second device 124 relative to a common fixed reference frame (earth, gravity) may be known, an angle of a joint (e.g., knee joint 78) coupling the first and second musculoskeletal portions (exemplified in FIG. 8 by a knee angle θknee) may be determined. Each of the first and second devices 122 and 124 may be calibrated based on an offset angle or calibration pose (e.g., when a person stands in a neutral anatomical position) to assist in measurement. For a knee joint 78, this calibration pose may occur at full extension of leg 62. Calibration may also be based on any known misalignments of femur 64 with respect to tibia 66.

The first and second devices 122 and 124 may include electronic circuitry and at least one sensor to measure orientation, pitch and/or roll of the sensor or a relative orientation, pitch, and/or roll between the sensors of first device 122 and second device 124. The sensors may include, for example, an inertial measurement unit (IMU), accelerometer, gyroscope, one or more strain gauges, etc. First device 122 and second device 124 may also include additional sensors or devices to obtain other data. For example, first and second devices 122 and 124 may include an external temperature sensor to sense temperature of skin, one or more internal temperature sensors to sense temperature of one or more components of the sensor itself, a communication module, a Bluetooth Low Energy (BLE) module, visual indicators (e.g., light emitting diodes or LEDs), a magnetometer (to determine absolute movements and orientations of the patient), a compass, a barometer, etc. First device 122 and second device 124 may also be configured to measure biometrics 1100 such as skin temperature, skin moisture, heartbeat, breathing rate, etc. First device 122 and/or second device 124 may include quantum dots, optical sensors, etc. First device 122 and/or second device 124 may be configured to remain coupled to the user's body for a day, two days, three days, four days, five days, six days, a week, two weeks, a month, or any other suitable time period.

The electronic circuitry in each of first device 122 and second device 124 may couple to at least one sensor. The electronic circuitry may be configured to control a measurement process and transmit measurement data, wirelessly or via one or more wires, to computer 21.

First device 122 and second device 124 may include a power source (e.g., battery, capacitor, cell such as a Lithium-ion cell, etc.), energy harvesting devices, and/or may be configured to receive power from an external source or commercial supply device (e.g., via wired or wireless connection, such as with wireless transceivers). The electronic circuitry may include power management circuitry configured to receive energy by inductive coupling, light coupling, or radio frequency coupling that is harvested and stored in first device 122 and/or second device 124 until sufficient energy may be stored to power first device 122 and/or second device 124 to complete a measurement.

Computer 21 may include one or more software programs to process measurement data received from first device 122 and/or second devices 124. Computer 21 may be any device having a processor, digital logic, a microprocessor, a microcontroller, a digital signal processor, a data conversion module, etc. that may be configured to support the software to process measurement data. For example, computer 21 may be a medical device, a phone, a tablet, a notebook computer, a personal computer, a robotic system, or a handheld device, among other examples. Computer 21 may have an application or “app” that is configured to direct a person through one or more movements to complete the process of registration for first device 122 and/or second device 124. Computer 21 may include visual, audible, or haptic feedback related to the registration process. A display of the computer 21 may provide visual feedback to support a person in real-time to complete the registration process, including instructions on how to perform the registration process as well as real-time feedback as the person performs the registration process.

Using data collected over time from first device 122 and second device 124, a change of the knee angle over a time period such as a day may be determined. The processed and calibrated data from each device 122, 124 can be passed to an orientation estimation unit, which may determine orientations of first device 122 and second device 124. In some implementations, the data and/or determinations made by the orientation estimation unit may include the pitch and roll undergone by first device 122 and second device 124. Calculated parameters such as pitch and roll data can be passed to a transmitter, such as an orientation data packing unit, for onward transmission. Such onward transmission could be by wired connections or unwired connections such as Bluetooth transmission or radio transmission etc. Computer 21 may include a processor, data conversion unit, a calibration unit, an orientation estimation unit, and/or the orientation data packing unit to analyze and transmit the data collected from first device 122 and second device 124.

Referring to FIG. 9, wearable sensors 114 may also be implemented as a tracking system 130 for a chest and/or shoulder 92. Tracking system 130 may include one or more devices 132 provided, for example, on a chest, torso, arm, leg, or back (e.g., surrounding shoulder 92). In some embodiments, tracking system 130 may include just one device 132 with only one IMU. Devices 132 may include any of the features of devices 122, 124 described with reference to FIG. 8. As an alternative to a leg 62 and/or shoulder 92, tracking systems 120 and/or 130 may be used to measure orientations, angles, alignments, acceleration, forces, etc. of other anatomical portions of the body such as a torso or pelvis area. For example, the tracking systems 120 and/or 130 may be used to measure or determine strength and/or force such as push-off power at a toe during walking. Tracking systems 120 and/or 130 may be used to measure orientations, angles, alignments, etc. of other joints, such as a hip joint, ankle joint, neck joint, etc. Tracking systems 120 and/or 130 may be used over a period of time to analyze data and assess balance or stability as a patient performs daily activities and/or activities that are routine for their lifestyle.

### Preoperative Outputs 7000

Referring to FIGS. 1 and 3, preoperative outputs 7000 may be determined via one or more preoperative algorithms 4000. Preoperative algorithms 4000 may also consider and/or analyze other previously stored data 50 of memory system 40 to determine preoperative outputs 7000. Preoperative outputs 7000 may include a prehabilitation plan 7010, a procedure, medical treatment, or surgical plan 7020, a postoperative plan 7030, a bone density score 7040, a fall risk or stability score 7050, a morphology score 7060, an EMG score 7070, an activity quality score 7080, a joint stiffness score 7090, a patient readiness score 7100, psychosocial score 7110, a b-score or bone shape score 7120, a push-off power score 7130, and a fracture risk score 7140. This list is not exhaustive, however. A “treatment course” or “course of treatment” may refer to any one of or all of prehabilitation plan 7010, procedure plan 7020, and postoperative plan 7030 and/or their intraoperatively determined and postoperatively determined analogs described later.

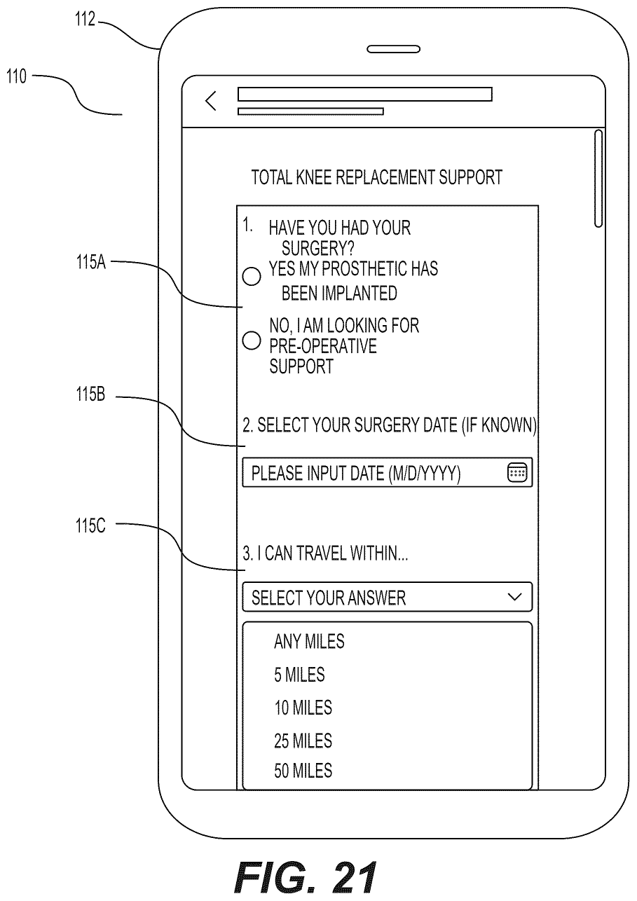

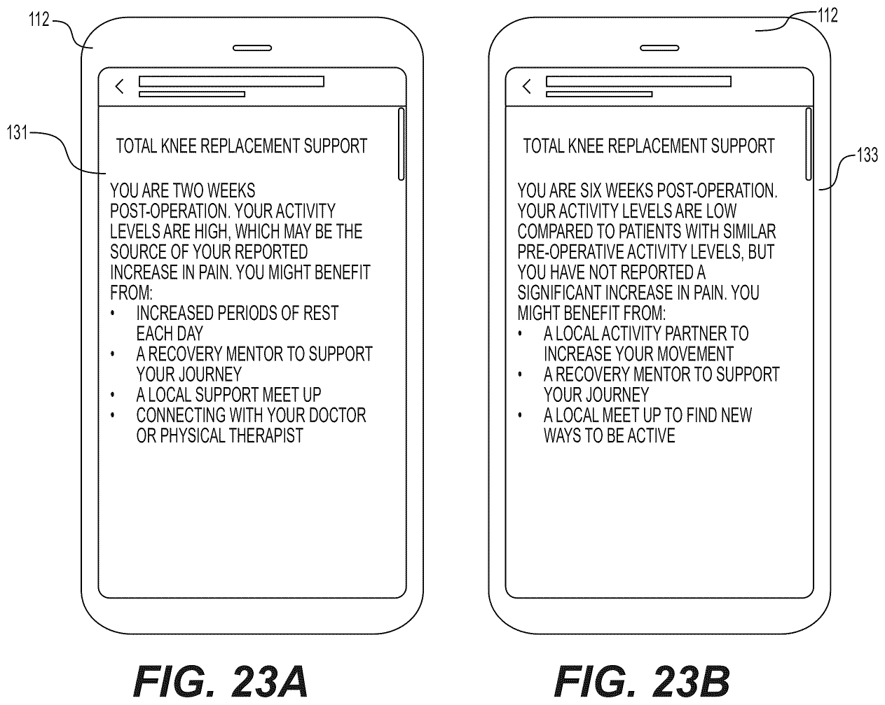

Prehabilitation plan 7010 may include instructions for a patient in preparing for a medical procedure or treatment course, such as surgery. For example, prehabilitation plan 7010 may include an exercise program which may include, a type of an exercise, a length of the exercise, a frequency of the exercise, or an order of a plurality of exercises. Prehabilitation plan 7010 may include a priority order of muscles to strengthen, etc. in preparation for the procedure. Prehabilitation plan 7010 may include other instructions or plans, such as medicine information (e.g., dosage and type) for the patient to take before the procedure. Prehabilitation plan 7010 may be configured to reduce a recovery time after the procedure. Prehabilitation plan 7010 may be based on one or more other postoperative outputs 7000, such as fall risk score 7050 and/or a stability score, bone density score 7040, activity quality score 7080, joint stiffness score 7090, patient readiness score 7100, psychosocial score 7110, b-score 7120, push-off power score 7130, fracture risk score 7140, etc. For example, patients with a higher fall risk score 7050, fracture risk score 7140, and/or a lower bone density score 7040 or push-off power score 7130 may need modified exercises. Specific embodiments of processes, algorithms, and/or feedback loops involving determinations by preoperative algorithms 4000, intraoperative algorithms 5000, and postoperative algorithms 6000 will be described later with reference to FIGS. 22 and 24.

Procedure, medical treatment, or surgical plan 7020 may include instructions for a surgeon in preparing for and/or performing a procedure (e.g., surgery) on the patient. For example, when procedure plan 7020 is a surgical plan 7020 for installation of an implant, the surgical plan 7020 may include, for example, a planned number, position, length, slope, angle, orientation, etc. of one or more tissue incisions or bone cuts, a planned type of the implant, a planned design (e.g., shape and material) of the implant, a planned or target position or alignment of the implant, a planned or target fit or tightness of the implant (e.g., based on gaps and/or ligament balance), a desired outcome (e.g., alignment of joints or bones, bone slopes such as tibial slopes, activity levels, or desired values for postoperative outputs 9000), a list of steps for the surgeon to perform, a list of tools that may be used, a planned operating room layout (e.g., positions and/or movement of objects or people in the operating room, such as staff, surgeons, medical or surgical robot 210, operating room table, patient, cameras, GUI, sensors, or other equipment), etc. Procedure plan 7020 may also include predictive or target outcomes and/or parameters, such as target postoperative range of motion and alignment parameters, target fall risk or fracture scores, activity quality scores, and joint stiffness scores. These target parameters may be compared postoperatively to corresponding measured postoperative data 3000 and/or determined postoperative outputs 9000 to determine whether an optimized outcome for a patient was achieved.

A design of the implant may include, for example, curvatures, shapes, or thicknesses and/or shimming parameters corresponding to a patient's anatomy (e.g., from bone imaging data 1080). For example, a design of the implant and/or prosthetic may be configured to match an arc of curvature of the implant with an arc of curvature of the native femoral condyle of the patient, an arc or curvature of a socket area or acetabulum, an arc or curvature of a glenoid or humerus, an arc or curvature of a tibial condyle, etc. Aspects disclosed herein may be applied to a custom knee implant design, custom hip implant design, custom partial knee or hip implant design, or custom design of any other implant design for any other part of a patient's anatomy. The design of the implant may also include materials of the implant and/or placement of implants of autologous tissue, allograft tissue, and/or synthetic materials. The design of the implant may include thicknesses, a number of shims configured to be stacked and/or removed, a size of an added shim, or other dimensions configured to adjust a fit or tightness of the implant.

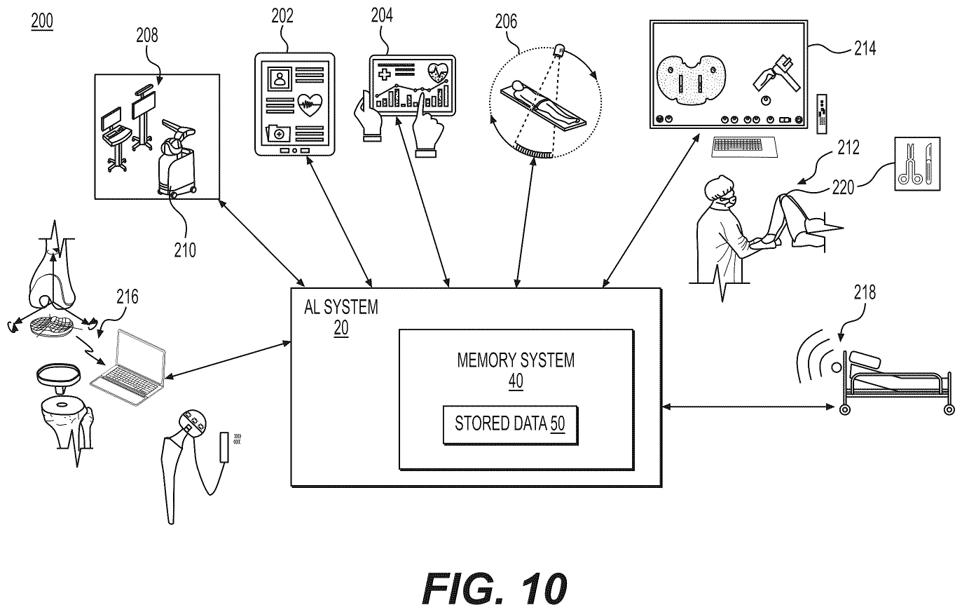

Procedure plan 7020 may also include instructions for a medical or surgical robot 210 to execute (see FIG. 10). Like prehabilitation plan 7010, procedure plan 7020 may be based on other preoperative outputs 7000. For example, in patients with a lower bone density score 7050 and a lower joint stiffness score 7090 (e.g., knee stiffness score), procedure plan 7020 may include an alignment of a tibial prosthetic with a lower tibial slope and/or a lower number of incisions.

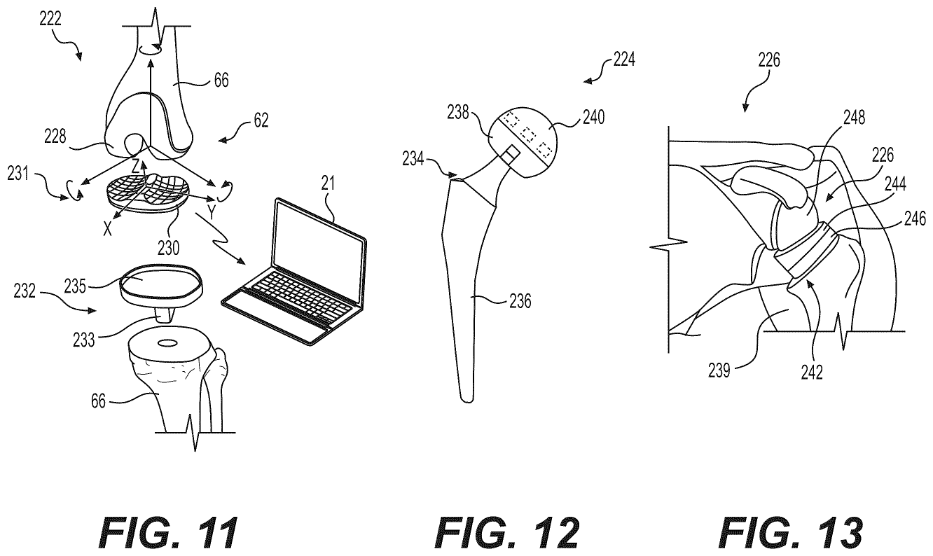

For example, with respect to FIG. 5 (see also FIG. 10), procedure plan 7020 may include instructions on how to prepare a proximal end of tibia 66 to receive a tibial prosthetic component 232 (FIG. 11), how to prepare a distal end of femur 64 to receive a femoral implant 228 (FIG. 11), how to prepare a glenoid or humerus to receive a glenoid sphere 248 and/or humeral prosthetic component 242 (FIG. 13), how to prepare a socket area or acetabulum to receive a ball joint 238 (FIG. 12), etc. The bone surface may be cut, drilled, or shaved relative to a reference (e.g., a transepicondylar axis). Bone cuts or drills to the femur and tibia may also be made referenced to vertical axis 84, mechanical axis 68, and/or anatomical axes 86, 90. The prepared bone surface may have a medial-lateral slope, anterior-posterior slope, and a compound slope configured to support accurate leg movement and proper rotation of the implant over a range of motion. Procedure plan 7020 may include positions lengths, and other dimensions for the surfaces and/or values for the slopes for bone preparation. As will be described later, procedure plan 7020 may be updated and/or modified based on intraoperative information 2000.

Postoperative plan 7030 may include plans similar to prehabilitation plan 7010 such as an exercise program configured to decrease a recovery time of the patient. Postoperative plan 7030 may further include a medication plan (e.g., pain medication plan including a type, dosage, and/or tapering of pain medication) and/or a discharge plan including a length of stay in a hospital. Postoperative plan 7030 may include a schedule of follow-up visits with a practitioner, surgeon, physical therapist, etc. Postoperative plan 7030 may also include a plan for revision surgeries or future additional surgeries, though procedure plan 7020 may be configured to reduce a likelihood of revision procedures or surgeries. Like prehabilitation plan 7010 and procedure plan 7020, postoperative plan 7030 may be based on other preoperative outputs 7000. For example, postoperative plan 7030 may include an exercise program configured to target muscles based on patient's lifestyle 1020 (e.g., frequency of climbing stairs or frequency of entering/exiting cars), fall risk score 7050, and/or fracture score 7140. Procedure plan 7020 may be updated and/or modified based on intraoperative information 2000 and postoperative information 3000.

Bone density score 7040 may be calculated from bone density data 1090, bone imaging data 1080 (e.g., morphology/anthropometrics data 1082), medical history 1030, and/or other information input by a patient or practitioner. Bone density score 7040 may be implemented as a T-score where a higher score correlates to a greater bone density, but aspects disclosed herein are not limited.

Fall risk score 7050 may be calculated from kinematics 1110, range of motion 1112 (e.g., postural sway), and alignment 1114. Fall risk score 7050 may be paired with or calculated based on lifestyle data 1020. For example, fall risk score 7050 may be calculated on a mobile device 108, be updated based on information sensed by mobile device 108 and be displayed on mobile device 108 (e.g., in a fall risk tracking app). Fall risk score 7050 may also be based on other preoperative outputs 7000 and/or qualitative observations or scores (e.g., frailty based on walking patterns or walking patterns assessed based on height and/or weight) and/or other observations input by a practitioner or patient (e.g., using EMR 102 and/or interfaces 104). A higher fall risk score 7050 may indicate a higher likelihood that a patient will fall or lose balance, or a higher frailty of the patient, but aspects disclosed herein are not limited.

Morphology score 7060 and/or a b-score or bone shape score 7120 may be calculated from bone imaging data 1080 and morphology/anthropometrics data 1082 using, for example, statistical shape modelling (SSM) or other processes. Morphology score 7060 and/or a b-score 7120 may also account for other data, such as alignment 1114, fractures, etc. “Machine-learning, MRI bone shape and important clinical outcomes in osteoarthritis: data from the Osteoarthritis Initiative” by Michael A. Bowes, Katherine Kacena, Oras A. Alabas, Alan D. Brett, Bright Dube, Neil Bodick, and Philip G. Conaghan, first published Nov. 19, 2020, explains details on calculating a b-score 7120 and is incorporated by reference herein in its entirety.

EMG score 7070 may be based on EMG data 1040 and may indicate an activity level of neurons and/or muscles. A higher EMG score 7070 may correspond to a higher level of activity, but aspects disclosed herein are not limited. Activity quality score 7080 may be based on lifestyle 1020, medical history 1030, EMG data 1040 and/or EMG score 7070, kinematics 1110, range of motion 1112, biometrics 1100, fitness level, and/or patient reported information. A higher activity quality score 7080 may indicate a higher activity level, activity quality, and/or fitness level of the patient, but aspects disclosed herein are not limited to a configuration or calculating of the activity quality score 7080.

Joint stiffness score 7090 may be calculated based on bone imaging 1080, kinematics 1110 (e.g., how quickly a patient can bend a joint), range of motion 1112, alignment 1114, etc. Each joint (e.g., knee, hip, ankle, neck) may have its own joint stiffness score 7090. A higher joint stiffness score 7090 may mean a higher stiffness and/or less laxity at the joint, but aspects disclosed herein are not limited.

Patient readiness score 7100 may be calculated based on psychosocial 1060 information (e.g., stress level) and/or psychosocial score 7010, biometrics 1100 (e.g., sleeping patterns), kinematic 1110, bone imaging 1080 etc. to assess a readiness for surgery. Patient readiness score 7100 may be updated or modified based on kinematics 1110, etc. measuring during performance of prehabilitation plan 7010, and prehabilitation plan 7010 may be updated and/or modified based on updated to patient readiness score 7100. As an example, biometrics 1100 indicated a decreased heart rate variability or HRV may indicate a higher level of stress and in turn a lower patient readiness score 7100.

Psychosocial score 7110 may be based on psychosocial 1060 information, such as stress, perceived pain, etc. and may also be based on biometrics 1100. Psychosocial 1060 information may be collected from surveys, practitioner observations, etc. A higher psychosocial score 7110 may indicate a higher level of stress, or alternatively may indicate a higher level of satisfaction, though aspects disclosed herein are not limited to a calculation of psychosocial score 7110. A decreased HRB may indicate a higher level of stress and in turn a higher psychological score 7110. Alternatively, psychosocial score 7110 may be configured to decrease based on a higher level of stress.

Push-off power score 7130 may be based on kinematics 1110, such as measured force, acceleration, contact pressure, etc. at a foot during walking (e.g., from a sensor in a shoe, coupled to the shoe, or coupled to the leg). A higher push-off power score 7130 may indicate a faster or stronger push-off during walking or spring in a step. Alternatively, or in addition thereto, the push-off score 7130 may be measured at the hands, such as during push-ups.

Fracture risk score 7140 may be calculated from kinematics 1110, range of motion 1112 (e.g., postural sway), bone density 1090, and alignment 1114. Fracture risk score 7140 may be paired with or be calculated based on lifestyle data 1020 and/or fall risk score 7050. For example, fracture risk score 7140 may be calculated on a mobile device 108, be updated based on information sensed by mobile device 108 and be displayed on mobile device 108 (e.g., in a fracture risk tracking app). Fracture risk score 7140 may also be based on other preoperative outputs 7000 and/or qualitative observations or scores (e.g., frailty based on walking patterns or walking patterns assessed based on height and/or weight) and/or other observations input by a practitioner or patient (e.g., using EMR 102 and/or interfaces 104). As an example, a lower bone density score 7040 and a higher fall risk score 7050 may result in a higher determined fracture risk score 7140. A higher fracture risk score 7140 may indicate a higher likelihood that a patient will fracture a bone, or a higher frailty of the patient, but aspects disclosed herein are not limited.

### Intraoperative Data 2000

Referring to FIGS. 2, 3, and 10, intraoperative data 2000 may include information taken during performance of a procedure plan 7020. Intraoperative data 2000 may include information on operating room efficiency 2010, procedure duration 2020, tourniquet time 2030, blood loss 2040, biometrics 2050, incision length 2060, soft tissue integrity 2070, pressure 2080, range of motion or other kinematics 2090, implant or prosthesis position 2100, and implant type or design 2110, though this list is not exhaustive. For example, intraoperative data 2000 may also include updated preoperative data 1000 (e.g., updated bone imaging 1080, etc.).

Operating room efficiency 2010 may include procedure duration information 2020, a number of practitioners performing the procedure plan 7020/8020, a number of medical or surgical tools used, etc. Operating room efficiency 2010 may also include information on an operating room layout, such as a room size, a setup, an orientation, starting location, and/or movement path of certain objects (e.g., surgical robot 210, practitioner, surgeon or other staff member, operating room table, cameras, GUI 214, other equipment, or patient). Cameras and/or a navigational system may be used to track operating room efficiency 2010 and/or layout information. Operating room efficiency 2010 may include information on staff and/or surgeon's performing the procedure plan 7020/8020, experience of each staff member or surgeon, past surgeries performed by each staff member or surgeon, and also scheduling information in an institution (e.g., hospital) where the surgery is taking place. Operating room efficiency 2010 may also include information on ergonomics for each staff member or surgeon, such as movement and posture patterns (measured by, for example, wearable sensors 114, external sensors, cameras and/or navigational systems, surgical robot 210, etc.) System 20 may make determinations to optimize operating room efficiency 2010. For example, based on ergonomics information, system 20 may determine that a table is too high for a surgeon and determine a lower height for the table in an updated operating room layout to include in the procedure plan 7020, which may increase operating room efficiency 2010 by reducing fatigue for a surgeon working over the operating table.

Procedure duration 2020 may include duration and/or other timing data of certain steps or procedures of the procedure plan 7020 and/or a total time of procedure plan 7020. Tourniquet time 2030 may include a time a tourniquet, cuff, or other restrictive device is applied to a limb. In addition, tourniquet time 2030 information may include pressure information at specific times or for specific time periods, where pressure information may be pressure applied to the limb, blood pressure, and/or pressure of, for example, an inflatable tourniquet. Blood loss 2040 may include information on an amount of blood lost during performance of procedure plan 7020. Biometrics 2050 may include all types of information included in preoperative biometrics 1110 and may also include other patient characteristics, such as temperature, heart rate, breathing rate, skin temperature, skin moisture, pressure exerted on the patient's skin, patient movement/activity etc. during performance of procedure plan 7020, etc. Incision length 2060 may include a length, position, and/or number of incisions actually made during performance of procedure plan 7020. Actual incision length 2060 may correspond to or be different from a predicted or planned incision length from data in planned procedure 1050 and/or procedure plan 7020.

Soft tissue integrity 2070 may include structural, strength, or density information for muscles, tendons, ligaments, and/or other soft tissue structures (e.g., skin) of the patient. Soft tissue integrity 2070 may be based on observed injuries (e.g., Posterior Cruciate Ligament or PCL injuries) during performance of procedure plan 7020 and/or based on prior observations. Soft tissue integrity 2070 may be an input and/or an output based on other preoperative inputs 1000 and intraoperative inputs 2000. Soft tissue integrity 2070 may be determined from a laxity assessment where a physician may stress a joint to determine tissue integrity. The laxity assessment may be a manual and subjective process, or alternatively may be controlled and/or quantified with sensors (e.g., wearable sensors 114, sensored implants 216) to measure applied force and/or joint displacement. For example, a practitioner may perform a varus/valgus stress test on a knee where a controlled force is applied to a shank to assess collateral ligaments. Diagnostic imaging systems 106 such as MRI scans may also be used to assess tissue integrity and/or to reveal structural or physiological changes. As another example, a practitioner may use a pendulum knee drop test (passive test) to determine overall stiffness or knee joint laxity. Soft tissue integrity 2070 may also be determined from bone density 1090, which may be determined from diagnostic imaging systems 106, as bone density 1090 may be correlated to ligament integrity and/or soft tissue integrity 2070.

Pressure 2080 may include information about a pressure or load (e.g., a contact pressure) applied to a patient's anatomy and/or a prosthetic component during performance of procedure plan 7020. For example, pressure 2080 may include information on a magnitude and a position or center of a load applied to a prosthetic component or implant (e.g., humeral component, glenosphere component, tibia component, femoral component, etc.). Range of motion 2090 may include similar information as preoperative range of motion 1112, although a surgeon may be manipulating a patient's body instead of the patient manipulating his or her own body. Intraoperative range of motion 2090 may include manipulation under anesthesia (MUA) data based on movements, exercises, stretches, and/or other manipulation performed by the surgeon to assess movement, release pain, and break up scar tissue.

Implant position 2100 may include information on an actual implant position or alignment during performance of procedure plan 7020. Actual implant position 2100 may correspond to or be different from a predicted or planned implant position from data in planned procedure 1050 and/or procedure plan 7020. Similarly, implant type 2100 may include information on an actual implant type, design, material, etc. during performance of procedure plan 7020. Actual implant type 2100 may correspond to or be different from a predicted or planned implant type in planned procedure 1050 and/or procedure plan 7020. For example, a practitioner may record a different implant type 2100 used for a procedure that is different from planned implant type 2100.

### Measurement System 200

Referring to FIGS. 2 and 10, system 20 may collect intraoperative data using intraoperative measurement system 200. Like preoperative measurement system 100, intraoperative measurement system 200 may include electronic medical records (EMR) 202, user interfaces or applications 204, and diagnostic imaging systems 206. Intraoperative measurement system 200 may also include a medical or surgical robotic system 208 including one or more robots 210, a sensored medical or surgical tool system 212, one or more sensored implants 216, and a sensored patient bed or operating table 218. EMR 202, user interfaces 204, diagnostic imaging systems 206, robotic system 208, robot 210, sensored tool system 212, motion sensor system 114, sensored implant 216, and sensored bed or table 218 of intraoperative measurement system 200 may each include one or more communication modules (e.g., Wi-Fi modules, Bluetooth modules, etc.) configured to transmit intraoperative data 2000 to memory system 40, system 20, to each other, etc.

System 20 may use EMR 202 to collect the same types of information as with preoperative EMR 102, and EMR 202 may include any of the features of preoperative EMR 102 discussed hereinabove. EMR 202 may also include updated records including intraoperative observations by one or more practitioners performing the procedure plan 7020. System 20 may use EMR 202 to collect and/or store operating room (OR) efficiency 2010, procedure duration 2020, tourniquet time 2030, blood loss 2040, biometrics 2050, incision length 2060, soft tissue integrity 2070, implant type 2110, etc.

System 20 may implement user interfaces 204 on electronic devices such as computers, tablets, and/or phones, for example via mobile applications and/or management websites or interfaces such as OrthologIQ®, to display and/or update intraoperative data 2000 or other relevant data as received. User interfaces 204 may present questionnaires, surveys, or other prompts for practitioners to enter information, such as information to update EMR 202, pressure data 2080, etc. These user interfaces 204 may communicate with one or more of the other devices in intraoperative measurement system 200 to display other data, such as pressure 2080 obtained from one or more pressure or load sensors (e.g. from surgical robot system 208, sensored surgical tool system 212, sensored implants 216, and sensored patient bed 318, etc.). User interfaces 204 may include graphical user interfaces (GUIs) 214 described in more detail later that may display intraoperative data 2000 and/or outputs 8000. These user interfaces 204 may be executed on other devices disclosed herein (e.g., using mobile devices or other computers).

Diagnostic imaging systems 206 may include computed tomography (CT) scans, magnetic resonance imaging (MRI), x-rays, etc. For example, just prior to starting a procedure and/or during performing procedure plan 7020, a fluorescence imaging system or other non-invasive imaging system may capture images of a patient's anatomy and update, in real time, these images (e.g., by displaying these images via GUI 214). Diagnostic imaging systems 206 may be used to collect and/or update, intraoperatively, bone imaging information 1080, including morphology and/or anthropometrics 1082 fractures, and bone density 1090.

Surgical robotic system 208 may include one or more surgical robots 210 configured to perform or assist with, via automated movement and/or sensing, at least a portion of procedure plan 7020. Surgical robot 210 may be implemented as or include one or more automated or robotic surgical tools, robotic surgical or Computerized Numerical Control (CNC) robots, surgical haptic robots, surgical tele-operative robots, surgical hand-held robots, or any other surgical robot. Surgical robot 210 may include or be configured to hold (e.g., via a robotic arm), move, and/or manipulate surgical tools and/or robotic tools such as cutting devices or blades, jigs, burrs, scalpels, scissors, knives, implants, prosthetics, etc. Surgical robot 210 may be configured to move a robotic arm, cut tissue, cut bone, prepare tissue or bone for surgery, and/or be guided by a practitioner via robotic arm to execute a procedure plan 7020,

Surgical robot 210 may include sensors (e.g., pressure sensors, temperature sensors, load sensors, strain gauge sensors, force sensors, weight sensors, current sensors, voltage sensors, position sensors, IMUs, accelerometers, gyroscopes, position sensors, optical sensors, light sensors, ultrasonic sensors, acoustic sensors, infrared or IR sensors, cameras, etc.) on one or more robotic arms, robotic tools or devices, or surgical tools; and may collect data during performance of procedure plan 7020 such as procedure duration 2020, biometrics 2050, pressure 2080, incision length 2060, implant position 2100, and/or implant position 2100. Data collected from surgical robot 210 may be referred to as robotic data.

Surgical robot 210 may include one or more wheels to move in an operating room and may include one or more motors configured to spin the wheels and also manipulate surgical limbs (e.g., robotic arm, robotic hand, etc.) to manipulate surgical or robotic tools or sensors. Surgical robot 210 may be a Mako SmartRobotics™ surgical robot, a ROBODOC® surgical robot, etc. However, aspects disclosed herein are not limited to mobile surgical robots 210.

Surgical robot 210 may be controlled automatically and/or manually (e.g., via a remote control or physical movement of surgical robot 210 or robotic arm by a practitioner). For example, procedure plan 7020 may include instructions that a processor, computer, etc. of surgical robot 210 is configured to execute. Surgical robot 210 may use machine vision (MV) technology for process control and/or guidance. Surgical robot 210 may have one or more communication modules (Wi-Fi module, Bluetooth module, NFC, etc.) and may receive updates to procedure plan 7020 and/or a new intraoperative surgical plan 8020 (described later with intraoperative outputs 8000). Alternatively, or in addition thereto, surgical robot 210 may be configured to update procedure plan 7020 and/or generate a new intraoperative surgical plan 8020 for execution.

Sensored surgical tool system 212 may include one or more sensored surgical tools 220 (e.g., a sensored marker). Sensored surgical tool 220 may be applied to or be worn by the patient during procedure plan 7020, such as a wearable sensor (e.g., wearable sensors 114), a surgical marker, a temporary surgical implant, etc. Although some surgical tools 220 may also be sensored implants 216 or surgical robots 210, other surgical tools 220 may not strictly be considered an implant or a robotic or automated device. For example, sensored surgical tool 220 may also be or include a tool (e.g., probe, knife, burr, etc.) used by medical personnel and including one or more optical sensors, load sensors, load cells, strain gauge sensors, weight sensors, force sensors, temperature sensors, pressure sensors, etc. System 20 may use the sensored surgical tool system 212 to collect data on pressure 2080, range of motion 2090, incision length 2060 and/or position, soft tissue integrity 2070, biometrics 2050, etc. Sensored surgical tool 220 may be or include a robotic handheld tool configured to be held in the surgeon's hand and automatically cut tissue or bone (and/or prepare tissue or bone for surgery) according to instructions from procedure plan 7020. For example, sensored surgical tool 220 may be or include a robotic burr, knife, or blade. The surgeon may hold a handle of sensored surgical tool 220, and sensored surgical tool 220 may execute instructions using feedback from sensors (e.g., for position and/or orientation) and using moveable or motorized tool heads (e.g., blade or knife head).

The one or more sensored implants 216 may include temporary or trial implants applied during procedure plan 7020 and removed from the patient during the surgical procedure, and/or permanent implants 216 configured to remain for postoperative use. Sensored implants 216 may include one or more load sensors, load cells, force sensors, weight sensors, current sensors, voltage sensors, position sensors, IMUs, accelerometers, gyroscopes, optical sensors, light sensors, ultrasonic sensors, acoustic sensors, infrared or IR sensors, cameras, pressure sensors, temperature sensors, etc. System 20 may use sensored implants 216 to collect data on range of motion 1112 (e.g., when the patient is manipulated by the surgeon during procedure plan 7020), biometrics 2050, pressure 2080, implant position 2100 (e.g., alignment), implant type 2110 (e.g., design, material), etc. The one or more sensored implants 216 may also be configured to monitor infection information. More details on sensored implants 216 are provided with reference to FIGS. 11-13.

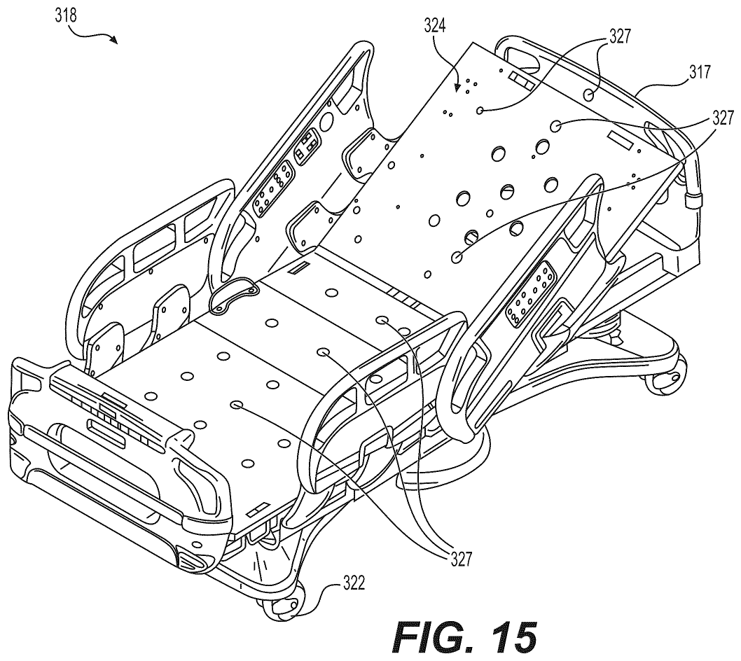

The one or more sensored patient bed or operating table 318 may be a bed or table including temperature sensors, load cells, pressure sensors, position sensors, accelerometers, IMUs, etc. System 20 may use sensored bed or table 218 to collect information on an orientation or position of the patient and biometrics 2050 (heart rate, breathing rate, skin temperature, skin moisture, pressure exerted on the patient's skin, patient movement/activity, etc.). Sensored bed or table 218 may include one or more wheels for movement, and sensored bed or table 218 may collect information on movement of bed or table 218, procedure duration 2020, etc. System 20 may implement sensored bed or table 218 as a postoperative sensored discharge bed 318 to sense patient movement and/or entrance/exit data. Postoperative sensored hospital or discharge bed 318 is described in more detail later with reference to FIGS. 15-16, and the sensored bed or table 218 may have a same or similar structure as the postoperative sensored discharge bed 318.

Referring to FIGS. 11-13, the one or more sensored implants 216 may be implemented as a knee prosthetic system 222, a hip prosthetic system 224, and/or a shoulder prosthetic system 226. Aspects disclosed herein are not limited to these types of knee, hip, and shoulder prosthetic systems 222, 224, 226. The one or more sensored implants 216 may be implemented as another implant system for another joint or other part of a musculoskeletal system (e.g., hip, knee, spine, bone, ankle, wrist, fingers, hand, toes, or elbow) and/or as sensors configured to be implanted directly into a patient's tissue, bone, muscle, ligaments, etc. Each of the knee, hip, and/or shoulder prosthetic systems 226 may include sensors such as inertial measurement units, strain gauges, accelerometers, ultrasonic or acoustic sensors, etc. configured to measure position, speed, acceleration, orientation, range of motion, etc. In addition, each of the knee, hip, and/or shoulder prosthetic systems 226 may include sensors that detect changes (e.g., color change, pH change, etc.) in synovial fluid, blood glucose, temperature, or other biometrics and/or may include electrodes that detect current information, ultrasonic or infrared sensors that detect other nearby structures, etc. to detect an infection, invasion, nearby tumor, etc. In some examples, each of the knee, hip, and/or shoulder prosthetic systems 226 may include a transmissive region, such as a transparent window on the exterior surface of the prosthetic system, configured to allow radiofrequency energy to pass through the transmissive region.

Prosthetic knee system 222 may include a femoral prosthetic component 228 configured to be coupled to a distal end of a femur 64, an insert 230, e.g., an alignment measurement device, and/or a tibial prosthetic component 232 configured to be coupled to a proximal end of a tibia 66. Femoral prosthetic component 228 may have one or more condyle surfaces (e.g., two condyle surfaces to mimic a natural femur). A design of tibial prosthetic component 232 may include predetermined numbers, sizes, shapes, materials (e.g., metal or metal alloy) of the condyle surfaces and femoral prosthetic component 228 as a whole. Insert 230 may be used to support installation of femoral prosthetic component 228 and/or tibial prosthetic component 232. Tibial prosthetic component 232 may include a tibial tray 235 and a tibial stem 233. A design of tibial prosthetic component 232 may include predetermined sizes (e.g., lengths), shapes, materials (e.g., metal or metal alloy) of tibial tray 235 and tibial stem 233. Tibial tray 235 may be configured to support and retain insert 230 to tibial prosthetic component 232, and tibial stem 233 may be configured to be inserted into a drilled hole in the tibia. For example, the tibial stem 233 may be configured to extend within the medullary canal of the tibia. Femoral prosthetic component 228 and/or tibial prosthetic components 232 may have one or more retaining features to couple to prepared bone surfaces of femur 64 and/or tibia 66, respectively.

Femoral prosthetic component 228, insert 230, and/or tibial prosthetic component 232 may include one or more sensors (e.g., within tibial tray 235 or within tibial stem 233) configured to measure kinematics and/or range of motion 2090 such as position, speed, acceleration, orientation, load, pressure 2080, force, and/or other parameters (e.g., biometrics 2050 such as temperature, pulse, blood pressure, bone density, colors or changes to synovial fluid to detect infection related data, blood glucose, heart rate variability, sleep disturbances, etc.). Alternatively, or in addition thereto, prosthetic knee system 222 may include one or more sensors coupled directly to femur 64 and/or tibia 66. In some examples, sensors positioned outside tibial prosthetic component 232, femoral prosthetic component 228, and insert 230 may be coupled to femur 64 and/or tibia 66 and may be in communication with electronic components within tibial prosthetic component 232, femoral prosthetic component 228, and/or insert 230.

Femoral prosthetic component 228, insert 230, and/or tibial prosthetic component 232 may include one or more inertial measurement units (IMU). For example, tibial stem 233 of tibial prosthetic component 232 may include a space or cavity to house one or more inertial measurement units (IMU). Alternatively, or in addition thereto, prosthetic knee system 222 may include an IMU coupled directly to femur 64 and/or tibia 66. The IMU may support real-time alignment measurement. The IMU may measure alignment of a leg 62 and support changes or modifications prior to final installation of femoral prosthetic component 228, insert 230, and/or tibial prosthetic component 232 to ensure alignment may be within a predetermined range for optimal performance and reliability.

The IMU may include three gyroscopes and three accelerometers, where a first, second, and third gyroscope and a first, second, and third accelerometer are respectively aligned to three perpendicular axes 231. Each gyroscope may measure an angular velocity corresponding to a rotation about an axis. In other examples, the IMU may include any number of gyroscopes and any number of accelerometers, may only include one or more gyroscopes and not include accelerometers, or may only include one or more accelerometers and not include gyroscopes. Each accelerometer may measure a change in motion (acceleration) corresponding to one of the axes. The IMU may include up to nine degrees of freedom (DOF), which may include accelerations, gyroscopic velocities, and magnetometer values for 3-dimensional space. For example, the IMU may include up to 9-DOF, 6-DOF, or 3-DOF, and is not limited to the above-described arrangement.

The IMU may include a micro-electromechanical (MEMs) integrated circuit. For example, one or more of the gyroscopes or accelerometers may be or include a MEMs integrated circuit. A form factor of a MEMs gyroscope integrated circuit or MEMs accelerometer integrated circuit may support placement in a prosthetic component or coupling to a prosthetic component or bone surface to measure alignment of the muscular-skeletal system. The MEMs gyroscope may have a resonating mass that shifts with angular velocity and output a signal corresponding to (e.g., proportional to) the angular velocity of the IMU. A MEMs accelerometer may have a mass-spring system that shifts in response to an exerted acceleration, e.g., counter to a bias of a spring in the mass-spring system.

Femoral prosthetic component 228, insert 230, and/or tibial prosthetic component 232 may include other sensors, such as strain gauge sensors, optical sensors, pressure sensors, load cells/sensors, ultrasonic sensors, acoustic sensors, resistive sensors including an electrical transducer to convert a mechanical measurement or response (e.g., displacement) to an electrical signal, and/or sensors configured to sense synovial fluid, blood glucose, heart rate variability, sleep disturbances, and/or to detect an infection in leg 62 and/or around the knee. Measurement data from the IMU and/or other sensors may be transmitted to a computer or other device of system 20 to process and/or display alignment, range of motion, and/or other information from the IMU. For example, measurement data from the IMU and/or other sensors may be transmitted wirelessly to a computer or other electronic device outside the body of the patient to be processed (e.g., via one or more algorithms) and displayed on an electronic display.

Hip prosthetic system 224 may include a femoral prosthetic device or component 234 including a femoral stem 236 configured to couple to a femur 64 (FIG. 10) of a patient and a ball joint or head 238 configured to couple to a hip bone. A design the of the femoral prosthetic device or component 234 may include a size (e.g., radius), shape, material, radius, etc. of femoral stem 236, ball joint 238 and/or a neck coupling stem 236 to ball joint 238. Hip prosthetic system 224 may have any of the features of knee prosthetic system 222 described with reference to FIG. 11.

Femoral prosthetic device 234 may include (e.g., within ball joint 238 and/or stem 236) one or more sensors 240 such as strain gauge sensors, IMUs, optical sensors, pressure sensors, load cells, ultrasonic sensors, acoustic sensors, and/or sensors configured to sense synovial fluid and/or detect an infection (e.g., via blood glucose, body temperature, sleep disturbances, heart rate variability, etc.). Femoral prosthetic device 234 may be configured to measure, via the one or more sensors 240, magnitude, location, and/or direction of forces placed on femoral prosthetic device 234 (e.g., on ball joint 238) and/or a position, orientation, speed, acceleration, etc. of femoral prosthetic device 234 (e.g., ball joint 238).

For example, the one or more sensors 240 may include three strain gauge sensors positioned circumferentially around a central circuit board of ball joint 238 and positioned at an equal distance from a center of ball joint 238 and/or some other reference. Each strain gauge sensor may be spaced equally from each adjacent sensor. Different strains, loads, pressures, forces, etc. measured by each strain gauge sensor may be processed to determine a load magnitude and location of the load applied to ball joint 238. Alternatively, or in addition thereto, femoral stem 236 may include sensors to measure, for example, rotation of the femur about the hip joint and/or ball joint 238 and/or whether femoral stem 236 is moving (e.g., loosely coupled to the femur, etc.). The measured strains and/or other data may be transmitted to system 20 or another computing platform to calculate load parameters, such as magnitude, location, direction, etc. of an applied load, force, etc. of a joint (e.g., hip joint) in real time, which may then be visualized on a display (e.g., via GUI 214 described with reference to FIGS. 14-18).

Referring to FIG. 13, shoulder prosthetic system 226 may include a glenoid prosthetic component or glenoid sphere 248, a humeral prosthetic component 242, and a measurement device or insert 244. Glenoid sphere 248 may be configured to be coupled to a prepared bone surface of a scapula 239, such as within glenoid cavity 241 of scapula 239. Glenoid sphere 248 may have an anchor or stem to support an attachment (e.g., via screws) to scapula 140. Glenoid sphere 248 has an external, convex curved surface configure to couple to measurement device 244. Humeral prosthetic component 242 may be configured to couple to a prepared bone surface of a humerus 243. Humeral prosthetic component 242 may have a humeral tray 246 configured to couple with measurement device 244. Measurement device 244 may have an external, concave curved surface configured to couple to the external, convex curved surface of glenoid sphere 248.

Glenoid sphere 248, humeral prosthetic component 242, and/or measurement device 244 may include at least one sensor such as strain gauge sensors, IMUs, optical sensors, pressure sensors, load cells, ultrasonic sensors, acoustic sensors, and/or sensors configured to sense synovial fluid and/or detect an infection (e.g., via blood glucose, body temperature, sleep disturbances, heart rate variability, etc.). The one or more sensors may be configured to measure, via the one or more sensors, magnitude, location, and direction of forces placed on glenoid sphere 248, humeral prosthetic component 242, and/or measurement device 244 and/or a position, orientation, speed, acceleration, etc. of glenoid sphere 248, humeral prosthetic component 242, and/or measurement device 244.

As an example, a plurality of sensors (e.g., strain gauge sensors, capacitors, and/or capacitive sensors, or IMUs) may be provided along a concave surface 245 of measurement device 244 which is contact with the convex surface of glenoid sphere 248. Alternatively or in addition thereto, the sensors and/or contact surfaces of the sensors may be raised (e.g., by 0.10 mm, 1 mm, 10 mm, etc.) with respect to a remaining portion of concave surface 245 such that glenoid sphere 248 contacts measurement device 244 only at the raised contact surfaces of the sensors. Measurement device 244 may include electronic circuitry configured to control a measurement process and transmit measurement data to memory system 40 and/or system 20 to be displayed on GUI 214. The shoulder joint may be taken through a range of motion, and the sensors in measurement device 244 may measure range of motion 2090. For example, a position of humerus 243, a load magnitude applied to measurement device 244 by glenoid sphere 248, and/or a contact point where glenoid sphere 248 couples to measurement device 244 can be measured and/or determined in real-time.

Although prosthetics are described with reference to FIGS. 11-13, sensored implants and/216 may also be implemented as implantable navigation systems. For example, sensored implant 216 may have primarily a sensing function rather than a joint replacement function. Sensored implant 216 may, for example, be a sensor or other measurement device configured to be drilled into a bone, another implant, or otherwise implanted in the patient's body.

### Intraoperative Outputs 8000

Referring to FIGS. 1 and 3, intraoperative outputs 8000 may be determined via one or more intraoperative algorithms 5000. Intraoperative algorithms 5000 may also consider preoperative data 1000 and/or outputs 7000 and/or other previously stored data 50 of memory system 40 to determine intraoperative outputs 8000. Intraoperative outputs 8000 may include an updated or new surgical plan 8020, an updated or new postoperative plan 8030, an updated or new bone density score 8040, an updated or new fall risk or stability score 8050, an updated or new activity quality score 8060, an updated or new joint stiffness score 8070, a patient readiness score 8080, an updated or new B-score 8100, and an updated or new fracture risk score 8140. This list is not exhaustive, however. For example, intraoperative outputs 8000 may also include some of preoperative information 7000 previously described.

As previously described herein, intraoperative algorithms 5000 may be used to generate and output surgical plan 8020. This surgical plan 8020 may be newly generated based on intraoperative outputs 8000 and/or may be a modification to procedure plan 7020 generated using preoperative information 7000 (and/or a manually input procedure plan 7020). For example, intraoperative algorithm 5000 may determine that only minor changes are necessary to update surgical plan 8020 based on range of motion 2090, biometrics 2050, actual incision length 2060 and/or implant position 2100 or type 2110, etc. As another example, a medical condition not known to a surgeon may not be apparent until intraoperative outputs 8000 is collected and analyzed (e.g., blood loss 2040, soft tissue integrity 2070, range of motion 2090, undetected bone fractures, etc.), and intraoperative algorithm 5000 may generate a new surgical plan 8020 accounting for the detected condition. Surgical plan 8020 may include the same types of information and/or parameters as preoperatively determined procedure plan 7020 (e.g., instructions on incisions, prosthetic type, etc.).

Referring to FIGS. 1, 3, and 14-18, during performance of procedure plan 7020 and/or 8020, GUI 214 may display intraoperative data 2000 and/or intraoperative outputs 8000 quantitatively, as graphs and/or tables, schematically, and/or visually as illustrations, animations, and/or videos. For example, GUI 214 may include or be implemented as GUI 214A, GUI 214B, 214C, 214D, or 214E, as shown in FIGS. 14-18, respectively. GUI 214 (e.g., GUI 214A, 214B, and 214D) may be configured to visualize or illustrate bones (e.g., femur 64 and/or tibia 66, humerus, scapula, hip joint, ankle joint, spine, etc.), prosthetic components or implants (e.g., sensored prosthetics and/or implants 216), and/or surgical tools 220 (e.g., markers) currently applied to and/or interacting with the patient's anatomy.

GUI 214 may also be configured to visualize (e.g., as a video, a virtual reality or VR platform, an augmented reality or AR platform, or a mixed reality or MR platform) real-time intraoperative data 2000 as its collected, such as range of motion 2090 from prosthetics and/or implants 216 and/or surgical tools 220 (e.g., as in 214A), alignment, positions, and/or orientations of prosthetics and/or implants 216, etc. (e.g., as in 214A, 214B, 214C, 214D, and 214E). GUI 214 may be configured to display real-time intraoperative data 2000 in multiple dimensions, such as 2D or 3D, and/or viewed with different mediums (e.g., a VR headset, an AR headset, or an MR headset) but not limited to the described devices. GUI 214 may be interactive so that a surgeon or other staff member may interact with displayed data in real-time intraoperatively.

In some embodiments, GUI 214 may be configured to display an optimized outcome of an alignment of prosthetics and/or implants 216 included in procedure plan 7020 (e.g., as in 214B or 214D), an updated optimized outcome included in surgical plan 8020 based on intraoperative data (e.g., as in 214B or 214D), bone shape (e.g., spine shape) (e.g., as in 214B or 214D), etc., and/or a real-time actual alignment, position, etc. on a same electronic screen to facilitate comparison (e.g., as in 214B). GUI 214 may be configured to display instructions, progress, and/or next steps of procedure plan 7020 and/or 8020, and any alerts or warnings based on certain determinations from intraoperative data 2000 and/or intraoperative outputs 8000 (low heart rate, high blood loss, etc.) and/or preprogrammed alerts or warnings (e.g., timing data, etc.) In some aspects, GUI 214 may display a determined operating room layout, schedule of medical personnel, workflows, etc. Any of the exemplified GUI 214A, GUI 214B, GUI 214C, GUI 214D, and/or GUI 214E may be displayed on an electronic screen, via one or more of the electronic devices discussed herein (e.g. a computer screen, mobile phone, tablet, surgical robot, etc.), either separately or at the same time as each other. GUI 214A, GUI 214B, GUI 214C, GUI 214D, and/or GUI 214E may be part of a surgical robotic system 208, part of a surgical robot 210, part of an operating room (OR) layout, part of a computer 21, etc.

Referring now to FIGS. 1 and 3, intraoperative outputs 8000 may include a postoperative plan 8030, which, like surgical plan 8020, may be newly generated based off of intraoperative outputs 8000 and/or may be a modification to postoperative plan 7030 generated using preoperative information 7000 (and/or a manually input procedure plan 7020). For example, based on range of motion 2090, biometrics 2050, actual incision length 2060 and/or implant position 2100 or type 2110, etc., postoperative plan 8030 may be modified to include recommended office visits, pain medications and dosages, a revision surgery, an exercise plan, etc. Postoperative plan 8030 may include the same types of information and/or parameters as preoperatively determined postoperative plan 8030 (exercise plan, discharge plan, pain medication plan, etc.).

Similarly, bone density score 8040, fall risk or stability score 8050, an activity quality score 8060, joint stiffness score 8070, B-score 8100, and fracture risk score 8140 may indicate (and be calculated from) similar information as preoperatively determined bone density score 7040, fall risk or stability score 7050, an activity quality score 7080, joint stiffness score 7090, B-score 7120, and fracture risk score 7140. Patient readiness score 8080 may, however, be an assessment of a readiness to end surgery and/or a readiness to discharge (rather than a readiness to have surgery), where a lower patient readiness score 8080 may indicate that more time is needed before ending surgery and/or discharging. Preoperative algorithms 4000, intraoperative algorithms 5000, and/or postoperative algorithms 6000 may calculate patient readiness score 8080 using procedure duration 2020 and/or blood loss 2040, in addition to similar parameters as patient readiness score 7100.

### Postoperative Data 3000

Referring to FIGS. 3-4, postoperative data 3000 may include information on patient outcome 3010, lifestyle 3020, patient satisfaction 3030, electromyography (EMG) 3040, planned procedures 3050 (e.g., revisions), psychosocial 3060, bone imaging 3080, bone density 3090, biometrics 3100, and kinematics 3110 including range of motion 3112 and/or alignment 3114, postoperative medical history 3129, and recovery 3130. This list, however, is not exhaustive and postoperative data 3000 may include other patient specific information and/or other inputs manually input by a practitioner. Some of postoperative data 3000 may be directly sensed, and other postoperative data 3000 may be determined (e.g., using a postoperative algorithm 6000) based on directly sensed or input information.

Patient outcome 3010 may include both immediate and long-term results and/or metrics from the medical procedure (e.g., surgery). For example, patient outcome 3010 may include a success metric or an indication of whether the procedure was successful, changes in range of motion, stability, fall risk or stability, fracture risk, joint stiffness or flexibility, or other changes between preoperative data 1000, or intraoperative data 2000 and postoperative data 3000, etc. Patient satisfaction 3030 may be a patient-reported (or, alternatively or in addition thereto, a practitioner-reported) satisfaction with the procedure, both immediate and long-term. Planned procedure 3050 may include information determined in outputting postoperative plan 8030 and/or other information on future planned procedures for the patient (e.g., a surgeon-created plan or revision based on patient outcome 3010, etc.) Medical history 3120 may include updated and/or new medical history 3120 (as compared to preoperative medical history 1030) and may include both immediate and long-term information such as new utilization of orthotics, care information in a supervised environment such as a skilled nursing facility or SNF, infection information, etc. Information on recovery 3130 may include information on adherence to a postoperative plan 8030 such as actual exercises performed, medicine dosage and/or type actually taken, fitness information, planned physical therapy (PT), adherence to PT, etc. Information on recovery 3130 may also include discharge and/or length of stay information.

Lifestyle 3020, EMG 3040, psychosocial 3060, QPM 3070, bone imaging 3080, bone density 3090, biometrics 3100, kinematics 3110, range of motion 3112, and/or alignment 3114 may include similar types of information as lifestyle 1020, EMG 1040, psychosocial 1060, bone imaging 1080, bone density 1090, biometrics 1100, kinematics 1110, range of motion 1112, and alignment 1114. For example, psychosocial 3060 may include perceived pain, stress, happiness, anxiety, etc.

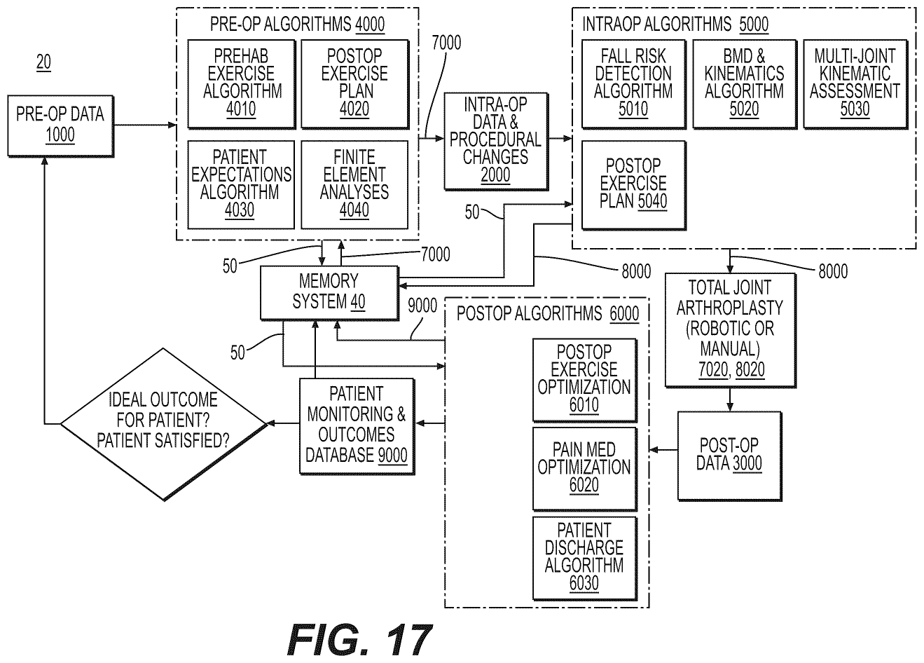

Psychosocial 3060 may include a quantitative pain metric (QPM) 3070. QPM 3070 may be derived from one or more data sources, including a facial classifier 3070(a) using facial expressions recorded by a mobile device camera, a movement classifier 3070(b) for evaluation of movement of a particular body part as recorded by the mobile device camera, and an implant movement classifier 3070(c). Further discussion of QPM 3070 is provided with respect to FIGS. 18-20.

### Postoperative Measurement System 300

Referring to FIGS. 2, 4, and 14, system 20 may collect postoperative data 3000 from postoperative measurement system 300. Like preoperative measurement system 100, postoperative measurement system 300 may include electronic medical records (EMR) 302, patient/user interfaces or applications 304, diagnostic imaging systems 306, mobile devices 308, a motion sensor and/or kinesthetic sensing systems 314, and electromyography or EMG systems 320. Like intraoperative measurement system 200, postoperative measurement system 300 may include one or more sensored implants 316, and a sensored patient bed 318. Devices implementing EMR 302, patient/user interfaces 304, diagnostic imaging systems 306, mobile devices 308, motion sensor system 314, EMG system 320, sensored implant 316, and sensored bed 318 of postoperative measurement system 300 may each include one or more communication modules (e.g., Wi-Fi modules, Bluetooth modules, etc.) configured to transmit postoperative data 3000 to memory system 40, system 20, to each other, etc.

EMR 302 may include any of the features of preoperative EMR 102 and intraoperative EMR 202 and may include updated records including postoperative observations by one or more practitioners performing procedure plan 7020. System 20 may use EMR 302 to collect information on postoperative medical history 3120, patient outcome 3010, lifestyle 3020, recovery 3130, planned procedures 3050, etc. Patient and/or user interfaces 304 may be similar to preoperative user interfaces 104. Patient interfaces 304 may present questionnaires, surveys, or other prompts for patients to enter psychosocial information 3060 such as perceived pain, stress level, anxiety level, feelings, and other patient reported outcome measures (PROMS). Patients may also report lifestyle information 3020 via patient interfaces 304. These patient interfaces 304 may be executed on other devices disclosed herein (e.g., using mobile devices 308 or other computers). Diagnostic imaging systems 306 may be similar to preoperative diagnostic imaging systems 106, and system 20 may use diagnostic imaging systems 306 to collect bone imaging information 3080, including morphology and/or anthropometrics, fractures, and bone density 3090.

Mobile devices 308 may include smartphones 310 or wearables 312 and be the same as or have any of the features of mobile devices 108 used preoperatively. System 20 may use mobile devices 308 to measure biometrics 3100, kinematics 3110, psychosocial information 3060, QPM 3070, lifestyle information 3020, etc. by including sensors that measure heart rate, electrocardiogram data, breathing rate, temperature, oxygenation, sleep patterns, activity frequency and intensity, and or by providing survey prompts and/or patient interfaces 304.

System 20 may use EMG systems 320 to collect EMG data 3040 and may be similar to preoperative EMG systems 116. Motion sensor and/or kinesthetic sensing systems 314 may be similar to preoperative motion sensor and/or kinesthetic sensing systems 114 and include motion capture (mocap) systems, external motion sensors, and wearable sensors to measure kinematics 3110 and range of motion 3112 data. Motion sensor and/or kinesthetic sensing systems 314 may include kinematics tracking systems which are the same or similar to kinematics tracking systems 120 and 130 used preoperatively. System 20 may use other types of stimulation systems (e.g., configured for a kinematic or EMG response) to collect postoperative data 3000.

Sensored implants 316 may be the same or include any of the features of permanent sensored implants 216 used intraoperatively. As an example, one or more temporary or trial implants 216 may be used intraoperatively to collect intraoperative data 2000, and a permanent implant 216 may be installed toward the end of the preoperatively determined surgical procedure 7020 and/or intraoperatively determined surgical procedure 8020. Postoperative implants 316 may be the same devices as permanent implants 216 installed during surgery intraoperatively. Like intraoperative sensored implants 216, the system 20 may use postoperative sensored implants 316 to collect kinematics 3110, range of motion 3112, and alignment 3114 (e.g., if an implant 316 becomes dislodged or misaligned). System 20 may also use sensored implants 316 to detect a presence of an infection or an infection rate at or near where the sensored implant 316 is installed by, for example, using sensors that detect changes in synovial fluid, blood glucose, body temperature, and/or using electrodes that detect current information, ultrasonic sensors that detect other nearby structures, etc.

Referring to FIGS. 14-15, postoperative sensored bed 318 (e.g., hospital bed, discharge bed, etc.) may be a moveable bed with multiple sensors to detect activity level and/or biometrics of a patient. Sensored bed 318 may include a bed frame or base 317 and a plurality of wheels 322, 323 to move bed frame 317. Plurality of wheels 322, 323 may include at least one front wheel 322 and at least one back wheel 323. As exemplified in FIGS. 14-15, the sensored bed 318 may include a pair of front wheels 322 and a pair of back wheels 323. At least one of front wheel 322 or back wheel 323 may be drive. For example, sensored bed 318 may include a motor to drive front wheels 322 for automated movement or transport, while back wheels 323 may be driven wheels.

Bed frame 317 may include a mattress frame or support 324 configured to receive a mattress 326 (FIG. 15). Mattress frame 324 and mattress 326 may be divided into sections corresponding to a patient's body (e.g., head area, torso area, pelvis area, thigh area, calf area, foot area, etc.), and each section may be pivotable with respect to adjacent sections for adjustment (by, for example, motors and/or actuators configured to drive links, rails, etc. coupled to or included in frame 317 and/or mattress frame 324). As shown in FIG. 15, each section and/or mattress frame 324 as a whole may also be raised and/or lowered using, for example, elevation adjusters 325 including actuators, pneumatic pumps, etc. Aspects disclosed herein are not limited to a design of frame 317 and/or mattress 326. As an example, U.S. Pat. No. 10,687,999 describes a sensored bed, which is incorporated herein by reference.

At least one of mattress frame 324 and/or mattress 326 may include a plurality of sensors 327, 329. The plurality of sensors 327, 329 may include a force sensor (e.g., load cell), optical sensor (e.g., laser sensor or infrared sensor), potentiometer, gyroscope-based sensor, accelerometer, magnetic sensor (e.g., Hall sensor or proximity sensor), a capacitive sensor, touch tape, a switch (e.g., a limit switch), etc. An arrangement of the plurality of sensors 327, 329 may be configured to measure loads, magnetic forces, capacitance, light, etc. at a plurality of positions. The arrangement of the plurality of sensors 327, 329 is not limited to the exemplary arrangement shown in FIGS. 15-16. These sensors 327, 329 may be used to measure biometrics 3100 (e.g., sleeping patterns, breathing rate) and kinematics 3110 (e.g., an amount of activity or movement, entrance/exit data, posture, and/or body alignment data, etc.). For example, mattress frame 324 may include a plurality of sensors 327, and mattress 326 may include a plurality of sensors 329. The plurality of sensors 327, 329 may be implemented as load cells provided at a plurality of positions in the various sections of mattress frame 324 and/or mattress 326 to determine a weight at a plurality positions, and from this data, an orientation of the patient's body may be determined, in addition to, over time, movement of the patient's body based on changes. Movement patterns may be used to determine sleeping patterns. Slight changes in movement may indicate breathing patterns.

Mattress frame 324 may also include sensors 329 configured to measure pulse or heart rate (e.g., upon contact of a patient's finger, etc.) The plurality of sensors may also be used to detect a moisture level on the skin, temperature, etc. Some of the data from the sensors may be used to determine psychosocial 3060 data (e.g., anxiety or stress data based on sleeping patterns), QPM 3070, or to calculate related postoperative outputs 9000 described later. Alternatively or in addition thereto, sensored pillows and/or bed sheets, quilts, etc. may be used with frame 317 and mattress 326. Data from these sensors may be combined with other wearable or attachable sensors used in hospitals to monitor patients (e.g., heart rate monitors, pulse oximeters, etc.).

### Postoperative Outputs 9000

Referring to FIGS. 1 and 3-4, postoperative outputs 9000 may be determined via one or more postoperative algorithms 6000. Postoperative algorithms 6000 may also consider preoperative information 1000 and/or outputs 7000, intraoperative information 2000 and/or outputs 8000, and/or other previously stored data 50 of memory system 40 to determine postoperative outputs 9000. Postoperative outputs 9000 may include an updated or new postoperative plan 9030, which may include a medication plan 9032 (e.g., for pain medication, antibiotics, etc.) and/or a discharge plan 9034, a patient readiness score 9010, an updated or new bone density score 9040, an updated or new fall risk or stability score 9050, an updated or new activity quality score 9060, an updated or new joint stiffness score 9070, an updated or new psychosocial score 9080, an updated or new B-score 9090, an updated or new push-off power score 9100, and an updated or new fracture risk score 9140. This list is not exhaustive, however. Updated or new bone density score 9040, fall risk or stability score 9050, activity quality score 9060, joint stiffness score 9070, psychosocial score 9080, B-score 9090, push-off power score 9100, or fracture risk score 9140 may include any of the features of preoperatively and intraoperatively determined bone density score 7040 and/or 8040, fall risk or stability score 7050 and/or 8050, activity quality score 7080 and/or 8060, joint stiffness score 7090 and/or 8070, psychosocial score 7110, B-score 7120 and/or 8100, push-off power score 7130, and/or fracture risk score 7140 and/or 8140, respectively. Patient readiness score 9010 may indicate a readiness to be discharged (rather than a readiness for surgery) and may be based on (and updated using) postoperative data and outputs 3000, 9000, such as patient outcome 3010, lifestyle 3020, patient satisfaction 3030, electromyography 3040, psychosocial 3060, bone imaging 3080, biometrics 3100, kinematics 3110, recovery 3130, fall risk score 9050, activity quality score 9060, psychosocial score 9080, push-off power score 9100, fracture risk score 9140, etc.

As previously mentioned, postoperative algorithms 6000 may be used to output postoperative plan 9030. This postoperative plan 9030 may be newly generated based on postoperative data 3000 and/or may be a modification to postoperative plan 8030 generated using intraoperative data 2000 (and/or a manually input) and/or postoperative plan 7030 generated using preoperative data 1000 (and/or manually input). In this context, for example, a medical practitioner may manually input an adjustment to postoperative plan 9030 via an electronic device. Postoperative plan 9030 may be continuously adjusted and/or updated as more postoperative data 3000 is collected.

As an example, postoperative algorithm 6000 may determine that only minor adjustments are necessary to update postoperative plan 8030 based on postoperative data 1000 like recover 3130, kinematics 3110, biometrics 3100, patient satisfaction 3030, lifestyle 3020, etc. As another example, unexpected responses or conditions indicated by postoperative data 1000, which may differ from expected or optimized postoperative conditions (e.g., increased or decreased perceived pain, lower or higher range of motion 3112, unexpected injury indicated in medical history 3120, etc.), may be analyzed and considered, and postoperative algorithm 6000 may generate a new postoperative plan 9030 (e.g., based on stored data 50 from other patients with similar unexpected conditions). Detailed determinations are described later with reference to FIGS. 22-33.

Postoperative plan 9030 may include any of the features of prehabilitation plan 7010 or procedure plans 7020, 8020, and/or 9020, such as an exercise program configured to decrease a recovery time of the patient. Postoperative plan 9030 may include a medication plan 9032 (e.g., pain medication plan including a type, dosage, and/or tapering of pain medication) and/or a discharge plan 9034 including a length of stay in a hospital. Medication plan 9032 may be based on psychosocial information 3060 (including QPM 3070) and may further be based on biometrics 3100 (e.g., heart rate variability and/or sleep patterns).

Postoperative plan 9030 may include any of the features of the preoperatively determined postoperative plan 7030, and may include a schedule of follow-up visits with a practitioner, surgeon, physical therapist, etc. Scheduled follow-up visits may be conducted remotely with markerless motion capture sensors and/or wearable sensors 114, sensored implants 216, etc. The scheduled follow up visit may be conducted via an application installed on mobile or remote devices 108. As explained later with respect to postoperative algorithms 6000, postoperative plan 9030 may be refined, generated, and/or updated throughout the postoperative period by postoperative algorithms 6000 based on postoperative data 3000 (e.g., kinematics 1110) and/or newly determined postoperative outputs 9000 (e.g., activity quality score 9060, etc.) obtained during scheduled follow-up visits or throughout the postoperative period. Refinement may occur at predetermined intervals, upon receiving new or predetermined postoperative data 3000, and/or continuously. The surgeon may review collected postoperative data 3000 and/or newly determined or updated postoperative outputs 9000 (which may be stored in memory system 40) without physically meeting with the patient. For example, if the patient has not yet reached predetermined goals two weeks after surgery, postoperative algorithms 6000 may update or determine a new postoperative plan 9030, and the practitioner may be notified of the update. The postoperative plan 9030 may also include a plan for revision surgeries or future additional surgeries, though procedure plan 7020 may be configured to reduce a likelihood of revision surgeries. Like surgical plan 8020, postoperative plan 9030 may be based on preoperative outputs 7000 and intraoperative outputs 8000. In addition, postoperative plan 9030 may be based on other postoperative outputs 9000. For example, postoperative plan 9030 may include an exercise program configured to target muscles based on patient's postoperative lifestyle 3020 (e.g., frequency of climbing stairs) and postoperatively determined fall risk score 9050 and/or fracture risk score 9140.

Medication plan 9032 may include instructions for pain medication or other medication (e.g., antibiotics). For example, medication plan 9032 may include a medication type, active ingredient, mechanism of action, route of administration, dosage level, dosage plan (e.g., taper plan of dosing), frequency, and/or other instructions related to taking medication. Medication plan 9032 may be based on postoperative data 3000 and postoperative outputs 9000 (and preoperative and intraoperative analogs) such as patient outcome 3010, lifestyle 3020, patient satisfaction 3030, planned procedure 3050, psychosocial 3060, bone imaging 3080, kinematics 3110, biometrics 3100, medical history 3120, recovery 3130, discharge plan 9034, patient readiness score 9010, psychosocial score 9080, etc. For example, medication plan 9032 may be based on a patient's prior drug history (collected from EMR 102, 302, etc.), perceived pain and/or PROMS (e.g., collected using apps or user interfaces 104 and/or 304), biometrics 3100 like heart rate variability and sleep patterns, bone imaging 3080 (e.g., fractures or healing of fractures), infections or sickness (e.g., detected from changes in synovial fluid using sensored implants 216 and/or 316, detected from sensors measuring blood glucose, body temperature, sleep disturbances, heart rate variability, etc.) and other recovery 3130 data.

Medication plan 9032 may be updated continuously and/or periodically postoperatively. For example, biometrics 3100 like certain heart rate variability patterns (e.g., higher heart rate) and/or short or infrequent sleeping patterns may indicate that the patient is experiencing a higher level of pain, and medication plan 9032 may be updated to increase a dose or determine a different (or stronger) type of pain medication. EMG data 3040 may also provide insight into pain levels. As another example, sensored implants 216 may detect information related to infections, and medication plan 9032 may be updated to include an antibiotic or other type of medication meant to treat the infection. Infection information may be sensed by sensored implants 216, or other sensors configured to detect a change in synovial fluid and configured to detect other biometrics 3100 such as heart rate variability, blood glucose, sleep disturbances, and body temperature which may indicate an infection at the surgical site. As another example, addictive behaviors may be determined (e.g., using biometrics 3100 and EMG data 3040 in combination with patient medical history or lifestyle 1020 and/or 3020 and/or other PROMS data), and medication plan 9032 may be created or updated to avoid and/or taper addictive pain medication, like opioids.

Discharge plan 9034 may include instructions for immediate recovery after surgery, such as a length of a hospital stay, supervision instructions, physical therapy instructions, target outputs (e.g., a fall risk threshold or target for fall risk score 9050, a target activity quality threshold or target for activity quality score 9060, a target patient readiness score 9010, a push-off power threshold or garget for push-off power score 9100, and/or a fracture risk threshold or target for fracture risk score 9140), etc. Discharge plan 9034 may be based on preoperative, intraoperative, and postoperative data and outputs 1000, 2000, 3000, 7000, 8000, an/or 9000. For example, discharge plan 9032 may be based on recovery 3130, medical history 3120, patient satisfaction 3030, patient outcome 3010, bone imaging 3080, kinematics 3110, biometrics 3100, fall risk score 9050, activity quality score 9060, bone density score 9040, patient readiness score 9010, push-off power 9100, and/or fracture risk score 9140.

For example, discharge plan 9034 (and/or patient readiness score 9010) may be updated using and/or based on postoperatively determined fall risk or stability score 9050 and/or postoperatively determined fracture risk score 91. Fall risk or stability score 9050 may be determined and/or updated using kinematics 3110 and biometrics 3100 using sensored hospital beds 318. Fracture risk score 9140 may be determined using fall risk score 9050 (or any inputs used to calculate fall risk score 9050) and bone density data 3090 and/or a determined bone density score 9040. Sensored hospital beds 318 may track entry/exit data, heart rate variability, sleep patterns, etc. Fall risk score 9050 and/or fracture risk score 9140 may increase, for example, based on certain (e.g., increased) heart rate combined with exit events (e.g., sensed using contact sensors on sensored hospital beds 318) and other kinematics data 3110 (e.g., acceleration data) from sensored implants 216. Based on an increased fall risk score 9050 and/or fracture risk score 9140, discharge plan 9034 may be updated to increase a number of days in the hospital.

### Processes and Systems

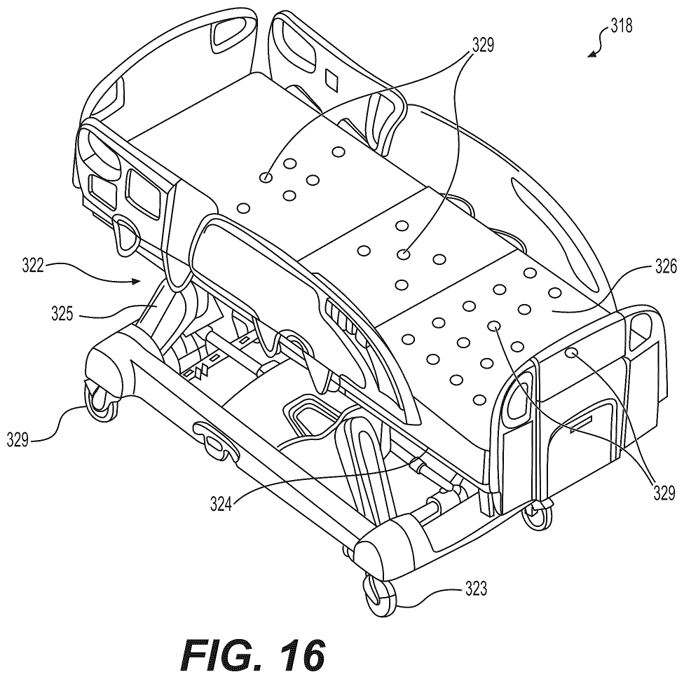

System 20 may be trained based on data from a plurality of patients and may be further trained and refined for each use for a specific patient. FIG. 17 illustrates a process flow diagram of system 20 executing preoperative algorithms 4000, intraoperative algorithms 5000, and postoperative algorithms 6000 in order to optimize outputs of system 20. For example, preoperative algorithms 4000 may include a prehabilitation (“prehab”) exercise program algorithm 4010, a postop exercise program algorithm 4020, a patient expectations algorithm 4030, and/or a finite element analysis algorithm 4040. Intraoperative algorithms 5000 may include a fall risk detection algorithm 5010, a bone mineral and/or marrow density (BMD) and kinematics algorithm 5020, a multi-joint kinematic assessment algorithm 5030, and/or a postop exercise program algorithm 5040. These algorithms 5010, 5020, 5030 may also be performed preoperatively, and may be included in preoperative algorithms 4000. Postoperative algorithms 6000 may include a postop exercise optimization algorithm 6010, a pain medication optimization algorithm 6020, and/or a patient discharge algorithm 6030.

Preoperative algorithms 4000, intraoperative algorithms 5000, and postoperative algorithms 6000 may implement machine learning and/or AI to be “trained,” or may learn and refine patterns between input information 10 and output information 30, which are used to make determinations.

Referring to FIGS. 1-4 and 17, may be executed utilizing system 20. Method 2200 may include a step 2202 of receiving preoperative data 1000 for an instant patient. Preoperative data 1000 may be received directly from preoperative measurement system 100 and/or from previously collected and stored data 50 in memory system 40. When preoperative data 1000 is received directly from preoperative measurement system 100, method 2200 may include a step 2204 of storing the received preoperative data into memory system 40. As previously described, the stored data 50 in memory system 40 may include all types of preoperative data 1000, intraoperative data 2000, and postoperative data 3000 from a plurality of previous patients

Referring to FIG. 18 and QPM 1070, an exemplary method 2300 of FIG. 18 may be executed utilizing system 20. Objective pain measurement (e.g., non-self-reported) may be a valuable metric to capture and analyze throughout a patient's preoperative and postoperative experience. Various methodologies may be employed to determine the amount of pain experienced by a patient. QPM 1070 may be a more reliable indicator than a self-reported pain score. Additionally, QPM 1070 may sometimes be the only reliable means of assessing patient pain if the patient is unable to communicate how they are feeling (for example, certain patients with developmental disabilities, surgery prevents the ability to speak or otherwise function, etc.).

During patient post-surgical rehabilitation (for example, following a total knee arthroplasty) with a smart knee and/or wearable or kinematic trackers (e.g., wearable 110, first device 122, second device 124, etc.), the patient may have access to an exercise plan (e.g., prehabilitation plan 7010), range of motion tracking (e.g., kinematics 1110), and a library of exercises to support their recovery. A patient may access this information via various devices described throughout this disclosure, including, but not limited to, wearable 110, smartphones 112, and computer 21. While discussion may be provided below with respect to a smartphone application, one of ordinary skill in the art will appreciate that the systems, devices, and methods of the disclosure are applicable to various other types of software and hardware.

In an example, a patient may complete an exercise or may move a body part (e.g., leg) through a range of motion. The motion and facial expressions of the patient may be tracked by smartphone 112 (e.g., via a camera), for example, which may both provide exercise instructions to the patient and record the exercise and facial expressions with a camera. It will be understood by one of ordinary skill in the art that the systems, devices, and methods of the disclosure may be applied to various other configurations of cameras and exercise instructions. For example, the patient may perform exercises (or other applicable information) shown on a display, and their movements and facial expressions may be captured by a separate camera (for example, in a lab setting, by a camera connected to a personal computer, or any other camera device or other sensor).

Captured (e.g., via a camera of smartphone 112) facial expressions may be analyzed to identify time intervals of pain and/or magnitudes of pain. Upon identification of periods of higher pain, the high-pain periods may be matched to kinematics 1110 and/or kinematics 3110) from either the smart knee (e.g., first and second devices 122 and 124) or wearable 110 trackers as well as what task/exercise the patient was performing. For example, a facial recognition algorithm may identify a period of high pain at a specific point during a knee flexion movement. System 20 may determine what the movement request was (e.g., knee flexion) and what the knee positioning was at that specific moment. This information may be used to report back to the patient on which exercises may be more helpful to their recovery, or if they should be avoiding certain exercises. For example, if system 20 detects high pain and unusually high rotation during active flexion, System 20 may suggest stepping back to a reduced range of motion and working to control rotation during this reduced movement. Conversely, if a patient shows little or no pain through a large range of motion, the software of system 20 (including AI system 21) may suggest more advanced exercises that may challenge the patient's movement and rehabilitation further.

The evaluated pain information (e.g., QPM 1070 and/or QPM 3070) may also be provided to system 20 to recommend an implant plan that aims to minimize pain for different patient phenotypes. Example activities/activities a patient may perform to capture full TKA kinematics may include a single-leg stance in full extension, a lunge, or a squat from mid-flexion to maximum flexion, kneeling from 90 degrees to maximum flexion, a chair-rise or stair-ascent, and standing open-chain rapid flexion-extension.

Following a TKA procedure, patients typically describe feelings of greatest instability during stair descent activities. A patient may describe a feeling indicative of instability, though this generally occurs after the feeling of instability has passed and is therefore not temporally linked to the unstable movement. During clinical rehabilitation, a camera may observe a patient descending stairs, such that the patient's facial expressions may be tracked during a stair descent activity. The patient's facial expressions during the stair descent activity may be analyzed to identify an expression of pain, uncertainty, and/or fear, indicative of instability. This facial expression may then be matched to the movement/activity at the time of the facial expression as well as immediately prior to the movement/activity. Additional data that may be captured include position, velocity, and/or acceleration of the tibia with respect to the femur. Characterizing instability in this manner may allow for enhanced implant positioning during surgery, in addition to informing implant designs to add constraints during points of instability.

This information (including any combination of any data associated with any of preoperative data 1000, intraoperative data 2000, and/or postoperative data 3000) may also be provided into System 20 to determine a stability safe zone (e.g., where the knee is neither too tight nor too loose) based on minimizing patient pain.

Quantitative pain metrics (QPMs) (e.g., via patient facial analysis) may more accurately and precisely gauge pain levels across a wide variety of patient phenotypes. Facial expressions, movements, and real-time data generated from wearables (e.g., wearable 110, wearable sensors 114) and/or smart implants (e.g., sensored implants 216) may correspond with videos and photos taken by a camera, such as a camera associated with smartphone 112. A QPM may correspond with evaluated pain intensity as determined by facial expressions, which may enable comparison between a patient's level of pain preoperatively and postoperatively. Additional comparisons may be made between robot assisted surgeries and manually performed surgeries. Various non-facial data sources may also be used when evaluating a QPM. For example, a movement classifier may correspond with body part movements during exercises/activities and may identify pain at the limits of a patient's range of motion and during expected movements. A smart-implant classifier may generate real-time data, such as temperature and patient-reported systems to distinguish between generalized and joint-specific infections and may correlate pain to the preoperative and postoperative time periods (e.g., how long it has been since a patient underwent surgery). Feedback may be provided to a patient based on the QPM, such as advising a patient (e.g., via a user device such as smartphone 112 running an application) to perform smaller movements if pain occurs at the limits of motion, reviewing exercise techniques if pain arises during expected movements (e.g., providing exercise form tips and feedback), and providing recommendations for medical consultation or rest based on the QPM and/or other symptoms.

FIG. 18 illustrates an exemplary method 2300 for preoperative and postoperative quantitative pain metric evaluation. Shown in method 2300 are a series of steps for preoperative pain classification of facial expressions (e.g., a preoperative classifier 2302) for pain evaluation. Also shown in method 2300 are a series of steps for postoperative evaluation of pain classification of facial expressions (e.g., a postoperative classifier 2312). The steps associated with both preoperative classifier 2302 and postoperative classifier 2312 may be substantially similar, and thus will be discussed concurrently (e.g., a step 2304 with a step 2314, etc.)

In a step 2304 (and a step 2412), electronic data processing system 1 may receive a video and/or image input of a patient's face while performing an activity. For example, a patient may perform a leg extension ten times while a camera captures their facial expressions. Preferably, steps 2304 and 2314 are performed under substantially similar conditions (e.g., the patient should perform the same activity/exercise/motion in both step 2304 and step 2314). In some aspects, a patient may record their activity with a smartphone in a home setting. In some aspects, a patient may be recorded in a clinical setting (for example, in a clinician's office) with a smartphone, professional camera, etc. One or more different videos may be simultaneously captured during steps 2304/2314. For example, a first video may capture a close-up of the patient's face while a second video may capture a wider frame to include the patient performing the activity. It will be understood that any number of videos/images may be taken.

In a step 2306 (and a step 2316) electronic data processing system 1 may perform preprocessing of the visual media data generated during step 2304 (or step 2314). Included in preprocessing step 2306 may be facial detection (e.g., recognizing that a particular portion of the visual media corresponds with a human face). Further included in step 2306 are one or more alignment operations of the one or more videos/images, including temporal alignment (e.g., ensuring that video frames or segments of the video a synchronized in time), spatial alignment (e.g., correcting for camera movement or lens distortion via techniques such as image registration), object alignment (e.g., consistently locating objects such as patient anatomy across frames), and feature alignment (e.g., tracking various key image points such as facial landmarks and joints across frames). Further included in step 2306 is normalization, including intensity normalization (e.g., histogram equalization, min-max normalization, and/or Z-score normalization), color normalization (e.g., white balance and color histogram matching), geometric normalization (e.g., aligning spatial properties of various frames, including rotations, scaling, and/or affine transformations), temporal normalization (e.g., frame rate conversion and/or temporal interpolation), and feature normalization to improve machine learning models.

In a step 2308 (and 2318) electronic data processing system 1 may perform texture analysis of video/image (e.g., visual media) input(s). Various characteristics and visual details may be included for texture analysis. For example, electronic data processing system 1 may perform texture extraction on frames from captured videos (e.g., patterns, granularity, regularity, and smoothness). Texture extraction may aid in various alignment operations described above. Texture analysis may include texture enhancement (e.g., visibility, noise, and clarity), texture segmentation (e.g., grouping portions of a visual media frame based on texture characteristics), texture mapping (e.g., applying captured 2D visual media texture data to a 3D model), and texture classification.

Further included in step 2308 (and 2318) is feature extraction of the visual media input. Electronic data processing system 1 may recognize one or more patient facial features, for example, such as eyes (including eye corners, pupil positions and eyelids), eyebrows (including eyebrow corners and eyebrow arches), nose (including nose tip, nostrils, and nasal bridge), mouth (including lip corners, upper and lower lip contours, and phitrum), jawline (including the chin and jaw corners), checks (including cheek contours), forehead, cars (including car position), and head pose (including pitch, yaw, and roll). Additional features may be extracted, including the upper body (including the neck, shoulders, and chest), arms (including elbows, wrists, and hands), torso (including spine curvature and hips), and lower body (including knees, ankles, and feet). Medical system 1 may recognize various features in videos/images such as joint angles, body segments, posture, alignment, gait, motion, repetition, and form (e.g., of a specific motion or exercise depicted in a video/photo). Medical system 1 may extract any of the aforementioned features, alone or in combination with any other aforementioned feature(s).

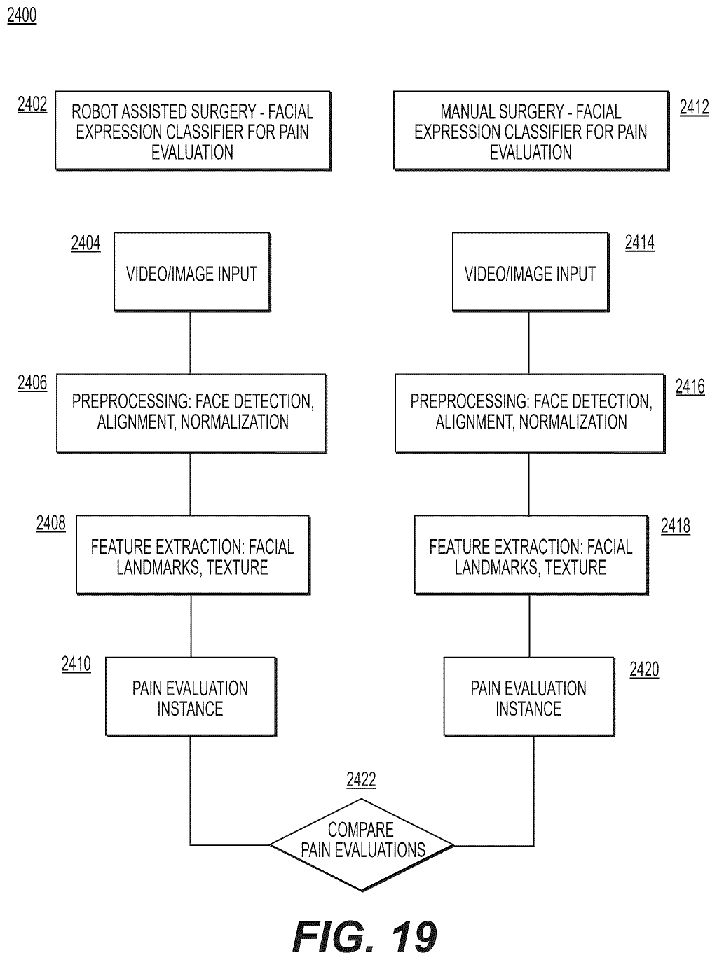

In a step 2310, electronic data processing system 1 may assign a QPM 1070 (similarly, in a step 2320, electronic data processing system 1 may assign a QPM 3070). Further discussion of evaluation/derivation of QPM 1070 (and QPM 3070) is provided with respect to FIG. 20.

In a step 2322, electronic data processing system 1 may compare pain evaluation instance 1 (e.g., step 2310) with pain evaluation instance 2 (e.g., step 2320). Various data may be compared, including facial features associated with specific movements and/or activities. For example, step 2322 may show reduced pain (as determined based on one or more facial features shown in one or more videos) in pain evaluation instance 2 (e.g., step 2320) compared to pain evaluation 1 (e.g., step 2310) for a given movement/activity. The compared pain evaluation may also be referred to as a quantitative pain evaluation score (QPES) 9150. One of ordinary skill in the art will appreciate that there may be various means of categorizing and evaluating QPES. For example, a patient may be expected to have a certain QPES associated with a positive patient outcome (e.g., based on a significantly lower pain evaluation instance 2). In another example, a patient experiencing a poor outcome may have a QPES indicative of similar (or worse) pain levels pre and post operatively (e.g., derived from steps 2310 and 2320). QPES may be derived and compared on an individual activity/movement basis. For example, a first activity may be associated with a first QPES and a second activity may be associated with a second QPES. One of ordinary skill in the art will appreciate that QPES may be used as a metric for associating, grouping, clustering, categorizing, organizing, classifying, sorting, aggregating, bundling, compiling, collecting, linking, connecting, aligning, matching, correlating, merging, combining, unifying, integrating, or joining one or more patients with similar QPESs. For example, it may be beneficial for a patient's recovery to communicate or perform rehabilitation activities with other patients that have a similar QPES. Various types of facial characteristics and/or features may be associated with the presence and/or intensity of pain. For example, a furrowed brow or grimace on a patient's face may be indicative of both the presence and intensity of pain. While these example are noted for discussion, any facial landmark may be used, alone or in combination with other facial landmarks, to be associated with the presence and/or intensity of pain.

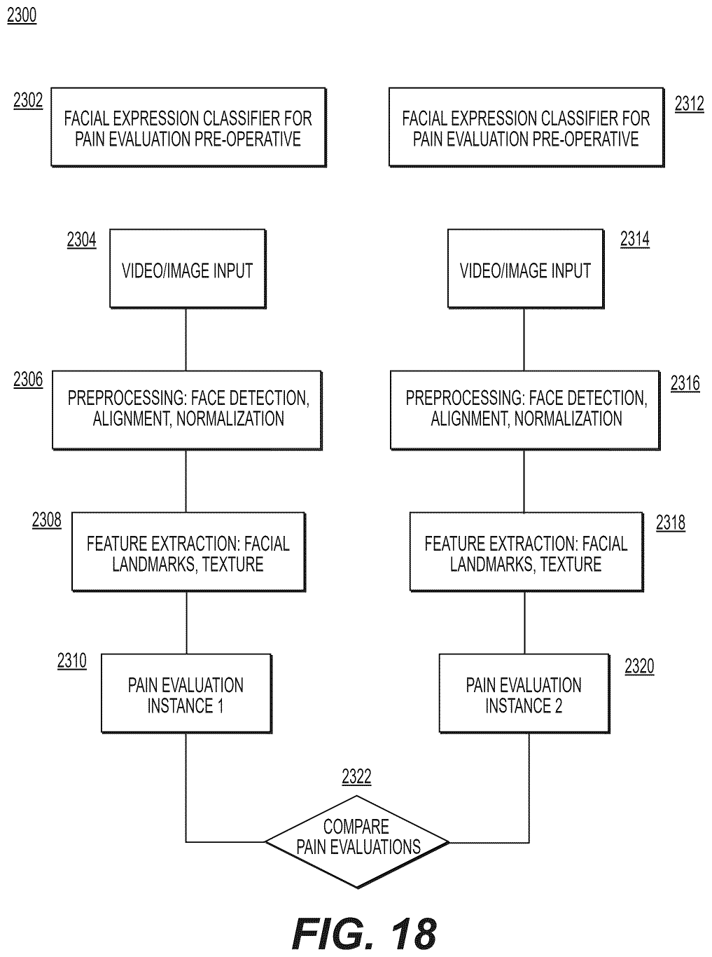

FIG. 19 illustrates an exemplary method 2400 for robot-assisted surgery and manual surgery quantitative pain metric evaluation. Shown in method 2400 are a series of steps for quantitative evaluation of pain via facial expressions following robot-assisted surgery (e.g., robot-assisted classifier 2402) for quantitative pain evaluation. Also shown in method 2400 are a series of steps for quantitative evaluation of pain following manual surgery via facial expressions (e.g., a manual classifier 2412). The steps associated with both robot-assisted classifier 2402 and manual classifier 2412 may be substantially similar, and thus will be discussed concurrently (e.g., a step 2404 with a step 2414, etc.) It may be clinical valuable to evaluate and compare patient pain in both the manual and robot-assisted context. Such evaluation and comparison may drive future clinical decision making (e.g., informing a choice of robot-assisted or manual surgery for a given type of surgery, patient characteristic, etc.).

In a step 2404 (and a step 2412), electronic data processing system 1 may receive a video and/or image input of a patient's face while performing an activity. For example, a patient may perform a leg extension ten times while a camera captures their facial expressions. Preferably, steps 2404 and 2414 are performed under substantially similar conditions (e.g., the patient should perform the same activity/exercise/motion in both step 2404 and step 2414). In some aspects, a patient may record their activity with a smartphone in a home setting. In some aspects, a patient may be recorded in a clinical setting (for example, in a clinician's office) with a smartphone, professional camera, etc. One or more different videos may be simultaneously captured during steps 2404/2414. For example, a first video may capture a close-up of the patient's face while a second video may capture a wider frame to include the patient performing the activity. It will be understood that any number of videos/images may be taken.

In a step 2406 (and a step 2416) electronic data processing system 1 may perform preprocessing of the visual media data generated during step 2404 (or step 2414). Included in preprocessing step 2406 may be facial detection (e.g., recognizing that a particular portion of the visual media corresponds with a human face). Further included in step 2406 are one or more alignment operations of the one or more videos/images, including temporal alignment (e.g., ensuring that video frames or segments of the video a synchronized in time), spatial alignment (e.g., correcting for camera movement or lens distortion via techniques such as image registration), object alignment (e.g., consistently locating objects such as patient anatomy across frames), and feature alignment (e.g., tracking various key image points such as facial landmarks and joints across frames). Further included in step 2406 is normalization, including intensity normalization (e.g., histogram equalization, min-max normalization, and/or Z-score normalization), color normalization (e.g., white balance and color histogram matching), geometric normalization (e.g., aligning spatial properties of various frames, including rotations, scaling, and/or affine transformations), temporal normalization (e.g., frame rate conversion and/or temporal interpolation), and feature normalization to improve machine learning models.

In a step 2408 (and step 2418) electronic data processing system 1 may perform texture analysis of video/photo input(s). Various characteristics and visual details may be included for texture analysis. For example, electronic data processing system 1 may perform texture extraction on frames from captured videos (e.g., patterns, granularity, regularity, and smoothness). Texture extraction may aid in various alignment operations described above. Texture analysis may include texture enhancement (e.g., visibility, noise, and clarity), texture segmentation (e.g., grouping portions of a visual media frame based on texture characteristics), texture mapping (e.g., applying captured 2D visual media texture data to a 3D model), and texture classification.

Further included in step 2408 (and step 2418) is feature extraction of the visual media input. Electronic data processing system 1 may recognize one or more patient facial features, for example, such as eyes (including eye corners, pupil positions and eyelids), eyebrows (including eyebrow corners and eyebrow arches), nose (including nose tip, nostrils, and nasal bridge), mouth (including lip corners, upper and lower lip contours, and phitrum), jawline (including the chin and jaw corners), cheeks (including check contours), forehead, cars (including car position), and head pose (including pitch, yaw, and roll). Additional features may be extracted, including the upper body (including the neck, shoulders, and chest), arms (including elbows, wrists, and hands), torso (including spine curvature and hips), lower body (including knees, ankles, and feet). Medical system 1 may recognize various features in videos/images such as joint angles, body segments, posture, alignment, gait, motion, repetition, and form (e.g., of a specific motion or exercise depicted in a video/photo). Medical system 1 may extract any of the aforementioned features, alone or in combination with any other aforementioned feature(s).

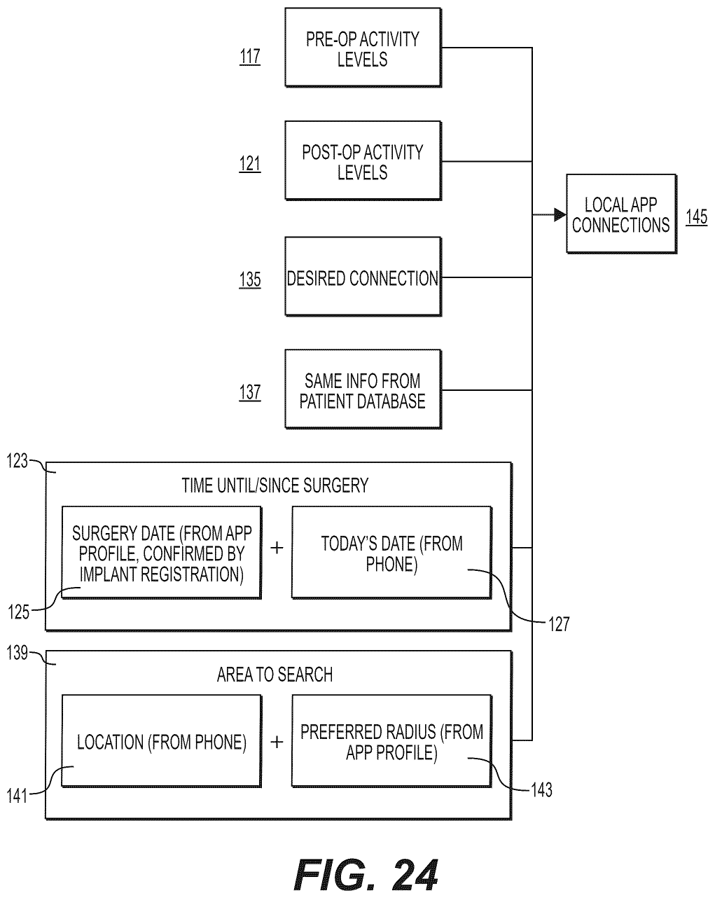

In a step 2410, electronic data processing system 1 may assign a QPM 1070 (similarly, in a step 2420, electronic data processing system 1 may assign a QPM 3070). Further discussion of evaluation/derivation of QPM 3070 (and QPM 1070) is provided with respect to FIG. 25.

In a step 2422, electronic data processing system 1 may compare pain evaluation instance 1 (e.g., step 2410) with pain evaluation instance 2 (e.g., step 2420). Various data may be compared, including facial features associated with specific movements and/or activities. For example, step 2422 may show reduced pain (as determined based on one or more facial features shown in one or more videos) in pain evaluation instance 2 (e.g., step 2420) compared to pain evaluation 1 (e.g., step 2410) for a given movement/activity. The compared pain evaluation may also be referred to as a quantitative pain evaluation score (QPES) 9150. One of ordinary skill in the art will appreciate that there be various means of categorizing and evaluating QPES. For example, a patient may be expected to have a certain QPES associated with a positive patient outcome (e.g., based on a significantly lower pain evaluation instance 2). In another example, a patient experiencing a poor outcome may have a QPES indicative of similar (or worse) pain levels for a given surgical method (e.g., derived from steps 2410 and 2420). QPES may be derived and compared on an individual activity/movement basis. For example, a first activity may be associated with a first QPES and a second activity may be associated with a second QPES. One of ordinary skill in the art will appreciate that QPES may be used as a metric for associating, grouping, clustering, categorizing, organizing, classifying, sorting, aggregating, bundling, compiling, collecting, linking, connecting, aligning, matching, correlating, merging, combining, unifying, integrating, or joining one or more patients with similar QPESs. For example, it may be beneficial for a patient's recovery to communicate or perform rehabilitation activities with other patients that have a similar QPES.

FIG. 20 illustrates an exemplary method 2500 for evaluation of a quantitative pain metric (e.g., QPM 1070 and/or QPM 3070). As previously discussed, QPM 1070 (and/or QPM 3070) may be based a facial expression classifier 2502, a movement classifier 2504, and a real-time sensor information classifier 2506, alone or in combination. Discussion of exemplary FIG. 25 with respect to use of facial expression classifier 2502, movement classifier 2504, and real-time sensor information classifier 2506 in combination, but one of ordinary skill in the art will appreciate that other combinations of these inputs are possible. It should be noted that in some aspects, a QPM may be based solely on facial analysis (e.g., facial expression classifier 2502, pain evaluation instances 2310, 2320, 2410, and 2420).

Facial expression classifier 2502 may be generated, for example, via steps 2304, 2306, 2308, and 2310. One of ordinary skill in the art will appreciate that other substantially similar steps shown in FIGS. 18 and 19 may also be used to generate facial expression classifier 2502 (e.g., the method of generating facial evaluation classifiers 2302, 2312, 2402, and 2412 is substantially similar).

Movement classifier 2504 may correspond with body part movements during exercises/activities and may identify pain at the limits of a patient's range of motion and during expected movements. Movement classifier 2504 may be based on any input information 10 or output information 30, alone or in combination of any subcomponent thereof. For example, movement classifier 2504 may be based on kinematics 1110, range or motion 1112, and alignment 1114, though this is only exemplary and other combinations of input data are within the scope of the disclosure.

Real time sensor information classifier 2506 may correspond with real-time data, such as temperature (e.g., via sensored implants 216) and patient-reported systems to distinguish between generalized and joint-specific infections, and may correlate pain to the preoperative and postoperative time periods (e.g., how long it has been since a patient underwent surgery). Feedback may be provided to a patient based on the QPM, such as advising a patient (e.g., via a user device such as smartphone 112 running an application) to perform smaller movements if pain occurs at the limits of motion, reviewing exercise techniques if pain arises during expected movements (e.g., providing exercise form tips and feedback), and providing recommendations for medical consultation or rest based on the QPM and/or other symptoms.

Facial expression classifier 2502, movement classifier 2504, and real-time sensor information classifier 2506 may be combined in a step 2508. The combination of the aforementioned inputs may assign various weightings to each input. For example, facial expression classifier 2502 may be weighed more than movement classifier 2504, or real time sensor information classifier 2506. The combined classifier output 2508 may be used to generate a quantitative pain metric (QPM) 2510, which may be used in various contexts such as those illustrated and described with respect to FIGS. 17-19. In an example, QPM 1070 may be included with preoperative data 1000, and QPM 3070 may be included with postoperative data 3000.

QPM 2510 may be evaluated to determine whether the amount of pain being experienced by a patient is relatively high. Various means of evaluation are possible. For example, QPM 2510 may be compared against a rubric defining a normal distribution of QPMs. The compared QPMs may be based on, for example, any input information 10 or output information 30, alone or in combination of any subcomponent thereof. For example, it may be desirable to compare QPMs of patients with similar demographics 1010 and kinematics 1110.

In a step 2514, feedback may be provided to a patient based on the QPM 2512, such as advising a patient (e.g., via a user device such as smartphone 112 running an application) to perform smaller movements if pain occurs at the limits of motion, reviewing exercise techniques if pain arises during expected movements (e.g., providing exercise form tips and feedback), and providing recommendations for medical consultation or rest based on the QPM 2512 and/or other symptoms.

In another aspect of the disclosure, AI-powered software configured to support patients before and after surgery through social connectivity and personalized guidance is disclosed herein. It is well known that patients may benefit from social interaction during surgical recovery, but certain patients, such as the elderly, may lack social networks needed to support their wellbeing. This problem is particularly pronounced for TKA recovery, where loneliness and insufficient social interaction may hinder patient recovery by reducing the patient's levels of physical activity.

To address this need, social recovery systems, devices, and methods are disclosed herein. Throughout this aspect of the disclosure, reference may be made to an application (e.g., a smartphone application). One of ordinary skill in the art will appreciate that this is only for ease of reference, and that the aspects of the disclosure may be applied to various types of hardware and software, such as smartwatches and desktop computers. Additionally, reference is made to an application in support of TKA recovery, but it will be understood that this aspect of the disclosure is applicable to various other types of surgical procedures. The application and underlying systems, devices, and methods disclosed in this aspect may connect a user who is planning to receive a TKA or is recovering from a TKA to a support network and may make intelligent suggestions to the patient based on various data.

The support network may include an activity partner, who may be an individual with similar activity levels and stage of recovery as the patient. The support network may include a recovery mentor, who may be an individual who has achieved a positive surgical outcome for a similar procedure as the patient. The support network may include an active meet-up, which may be a local gathering of similar patients (as matched by the systems, devices, and methods of the disclosure) to perform activities together. The support network may include group physical therapy, including local options (which may present cost savings to the patient and/or provider). The support network may include a chat functionality, including an online message board or forum configured to allow patients to connect with one another. The support network may include a non-patient partner function, which may match the patient to an individual that helps that patient stay committed to their recovery goals. The support network may include a suggestion for support feature that may be configured to recommend one or more support network components to a patient.

The systems, devices, and methods of the disclosure are primarily applicable to preoperative and postoperative scenarios. In the preoperative scenario, the patient may connect a wearable 110 or manually track their activity levels to the application and/or system. The application may allow the patient to create a personal profile, select one or more activities that they prefer (e.g., walking, swimming, or playing cards), and connect with local peers who have similar surgery dates and activity levels. As previously noted, patients may also seek advice through the application from recovery mentors who have already undergone surgery. System 20 may evaluate data such as date of surgery, location, preoperative activity levels, and desired connections (e.g., an activity partner and/or recovery mentor) to suggest potential matches. In some aspects, both the patient and the connectee (e.g., an activity partner and/or recovery mentor) must both accept one another as a match to connect.

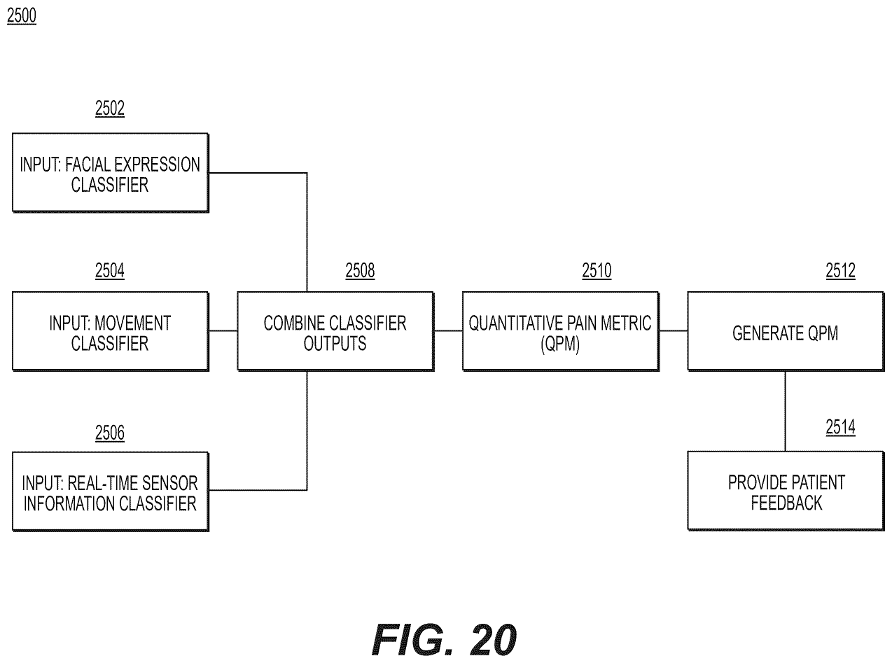

In the postoperative scenario, patients may enter specific profile information (shown in FIG. 21), which may include period pain scores (including both user-entered scores as well as QPMs) and activity levels (including both manual entry and automatic tracking via wearable 110). As previously noted, the systems, devices, and methods provide personalized suggestions and local connections for a user or personalized suggestions. A patient matching algorithm may compare the patient's postoperative activity levels to a database of other patients with similar profiles. For example, a patient that is two weeks post-surgery who was previously active for two hours daily but is now active for 30 minutes may be compared with other patients in the database who had similar preoperative activity levels. The algorithm also considers patient-reported pain scores and/or QPMs, regardless of preoperative activity levels, to accurately evaluate recovery impacts.

FIG. 21 illustrates an exemplary user interface 115A-115C for a patient recovery application 113. Application 113 may be executed and displayed on smartphone 112, though one of ordinary skill in the art will appreciate that application 113 may run on a variety of devices such as wearables, laptops/desktops, tablets, etc. It will further be appreciated that application 113 may be a web application (e.g., a web app). A user interface element 115A may include a prompt for a patient to answer whether they have had surgery or not (e.g., defining a preoperative or postoperative scenario). A user interface element 115B may include a prompt for a user to enter a date of surgery (whether future-facing or retroactively). Finally, a user interface element 115C may include a prompt for a user to enter a travel preference, which may include how far they are willing to travel (e.g., defining the radius of potential patients to connect with).

FIG. 22 illustrates an exemplary flow chart for generating personalized suggestions to a patient. System 20 may evaluate preoperative activity levels 117. Preoperative activity levels 117 may include kinematics 1110, range of motion 1112, alignment 1114, activity quality score 7080, joint stiffness score 7090, and/or push-off power 7130. In some aspects, preoperative activity levels 117 may incorporate any preoperative data 1000 and/or preoperative outputs 7000.

System 20 may evaluate pain scores 119. Pain scores 119 may include psychosocial 1060, psychosocial 3060, QPM 1070, QPM 3070, QPES 9150, psychosocial score 7110, and/or psychosocial score 9080. It should be understood that in some aspects of the disclosure, pain score 119 may depend solely on QPM 1070, QPM 3070, and/or QPES 9150. It will be apparent that pain scores 119 may include both objective (e.g., a QPM) and subjective (e.g., self-reported) pain metrics.

System 20 may evaluate postoperative activity levels 121. Postoperative activity levels 121 may include kinematics 3110, range of motion 3112, alignment 3114, activity quality score 9060, joint stiffness score 9070, and/or push-off power 9100. In some aspects, postoperative activity levels 121 may incorporate any postoperative data 3000 and/or postoperative outputs 9000.

System 20 may evaluate a time until/since surgery 123. Time until/since surgery 123 may include a surgery date 125. Surgery date 125 may be entered by a user to the application via user interface 115A, shown in FIG. 21. Surgery date 125 may be confirmed by a user, such as a surgeon, by a registration associated with a given implant (e.g., femoral prosthetic device 234) for a given procedure. Time until/since surgery 123 may also be referred to as a time interval.

Finally, system 20 may evaluate preoperative activity levels 117, pain scores 119, postoperative activity levels 121, and time until/since surgery 123 to generate suggestions 129 for improved patient outcomes. For example, application 113 may provide a patient personalized suggestions, via a suggestion algorithm that will compare the patient's postoperative activity levels to a patient database of patients' postoperative activity levels at the similar timeframe for patients with similar preoperative activity levels.

For example, a patient that is two weeks postoperative and typically had two hours of daily active time preoperatively, and is presently active for about 30 minutes per day may be compared against a patient database pool of patients with approximately similar preoperative and postoperative activity levels. In addition, the suggestion algorithm may assess how patient-reported pain scores and/or QPMs may be impacting recovery, regardless of preoperative activity level.

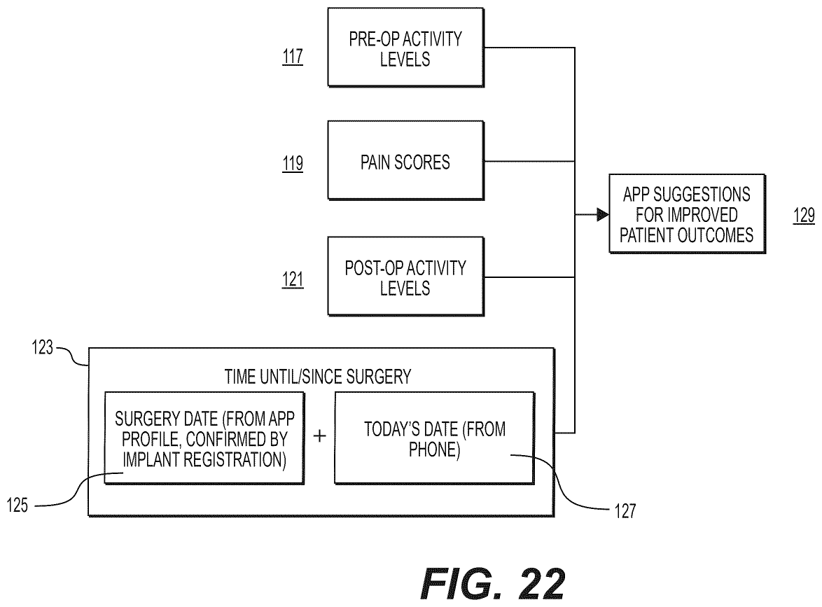

FIGS. 23A-23B show exemplary user suggestions (e.g., suggestion 129) provided via application 113 on smartphone 112. FIG. 23A shows a first suggestion, illustrated by user interface element 131. User interface element 131 may include a suggestion generated via inputs discussed with respect to FIG. 22. In the exemplary FIG. 23A, user interface element 131 may inform the patient that they are two-week post operation (e.g., time until/since surgery 123), and that their activity levels are high (e.g., postoperative activity levels 121), which may be the cause of the patient's reported increase in pain (e.g., pain score 119). User interface element 133 may include a suggestion generated via inputs discussed with respect to FIG. 22. In the exemplary FIG. 23B, user interface element 133 may inform the patient that they are six weeks post operation (e.g., time until/since surgery 123) and that their activity levels are low (e.g., postoperative activity levels 121). User interface element 133 may inform the patient that an increase in physical activity may be beneficial to recovery, as the patient has not reported an increase in pain (e.g., pain score 119). User interface element 133 may suggest local activity partners, recovery mentors, and local meet ups to the patient to encourage additional physical activity.

FIG. 24 illustrates an exemplary flow chart for generating local connections (e.g., activity partners, recovery mentors, and/or recovery mentee). System 20 may evaluate preoperative activity levels 117. Preoperative activity levels 117 may include kinematics 1110, range of motion 1112, alignment 1114, activity quality score 7080, joint stiffness score 7090, and/or push-off power 7130. In some aspects, preoperative activity levels 117 may incorporate any preoperative data 1000 and/or preoperative outputs 7000.

System 20 may evaluate postoperative activity levels 121. Postoperative activity levels 121 may include kinematics 3110, range of motion 3112, alignment 3114, activity quality score 9060, joint stiffness score 9070, and/or push-off power 9100. In some aspects, postoperative activity levels 121 may incorporate any postoperative data 3000 and/or postoperative outputs 9000.

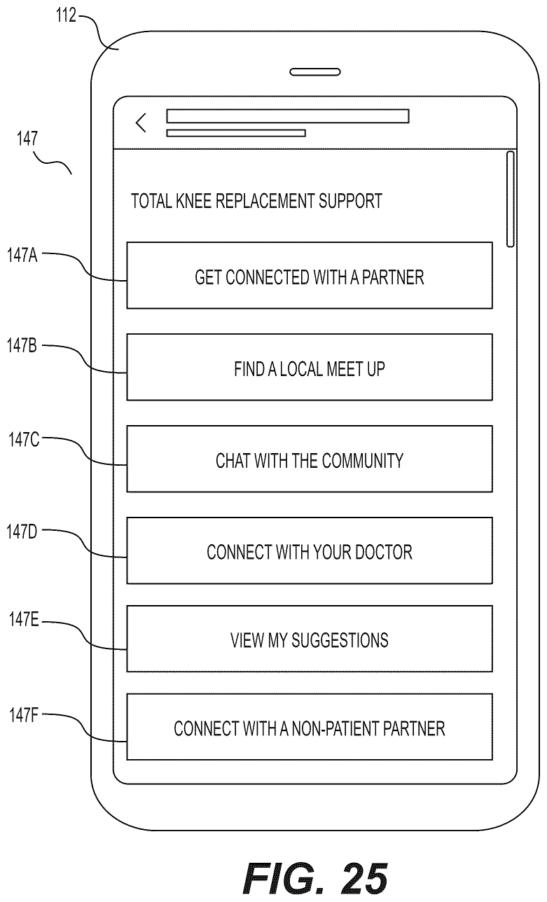

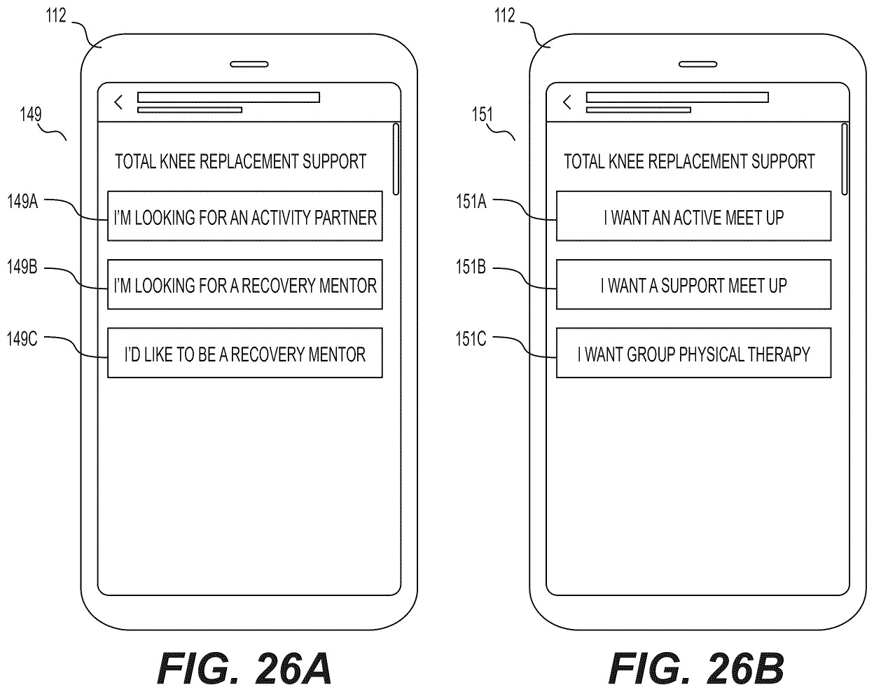

System 20 may evaluate a desired connection 135. Application 113 may offer (as shown in FIG. 26A) various connection options for a patient. These connection options may include finding an activity partner, recovery mentor, recovery mentee. When searching for an activity partner, application 113 may match the patient with a nearby individual who shares similar activity levels (e.g., preoperative activity levels 117 and/or postoperative activity levels 121) and is at a comparable stage of recovery, based on the patient's profile and current location. In scenarios where the patient is looking for a recovery mentor, application 113 (via System 20) may identify a local mentor who has volunteered for the role, prioritizing mentors with higher activity levels, lower pain scores, and/or more advanced recovery stages. Similarly, if the patient is seeking a recovery mentee, application 113 may identify someone locally who is looking for a mentor.

Further included in desired connection 135 are community connections (shown in FIG. 26B), System 20 (via one or more matching algorithms) may search the event and community group database to match the patient's desired community connections such as in-person meet-ups, virtual community groups, or group physical therapy sessions. Individual patients may create public meet-up events or groups linked to a specific location. Application 113 (via System 20) may identify local meet-ups that align with the patient's activity level or recovery period for active meet-ups. For support meet-ups, application 113 may group users based on pain and activity levels, if applicable. When seeking virtual community groups, application 113 may identify public groups with members who share similar activity levels, interests, or recovery periods. For group physical therapy, application 113 may identify local sessions organized by members' reported pain levels, activity levels, and postoperative stages.

System 20 may evaluate same and/or similar information from a patient database. One of ordinary skill in the art will appreciate that various mechanisms of data storage and access may be utilized, including SQL databases, non-SQL databases, time-series databases, object-oriented databases, multimodal databases, distributed databased, and the like. The patient database may include any preoperative data 1000, preoperative output 7000, intraoperative data 2000, intraoperative output 8000, postoperative data 3000, postoperative output 9000, and any data associated with any user of application 113.

System 20 may evaluate a time until/since surgery 123. Time until/since surgery 123 may include a surgery date 125. Surgery date 125 may be entered by a user to the application via user interface 115A, show in FIG. 21. Surgery date 125 may be confirmed by a user, such as a surgeon, by a registration associated with a given implant (e.g., femoral prosthetic device 234) for a given procedure.

System 20 may evaluate an area to search 139. Area to search 139 may include a location 141, which may be derived based on a current location of smartphone 112. Various means of location 141 are within the scope of this disclosure, including GPS, Wi-Fi positioning, cell tower triangulation, Bluetooth positioning, and IP address location. Area to search 139 may include a preferred radius 143, which may be derived by a user input to user interface element 115C of application 113.

System 20 may evaluate all of the aforementioned inputs to generate a list of one or more local connections 145. Local connections 145 may be ordered by relevancy, with the strongest matches presented more prominently (e.g., higher) in the list than less relevant matches. System 20 may be configured to not present matches to a patient that fall below a predefined threshold, thereby filtering irrelevant results. For example, System 20 may exclude potential mentors with lower postoperative activity levels 121 than a patient.

FIG. 25 shows an exemplary user interface 147 provided via application 113 on smartphone 112. User interface element 147 may include a user interface element 147A, which may be interacted with by a patient to bring the patient to partner connection interface (shown in FIG. 26A). User interface element 147 may include a user interface element 147B, which may be interacted with by a patient to bring the patient to a meet up interface (shown in FIG. 26B). User interface element 147 may include a user interface element 147C, which may be interacted with by a patient to bring the patient to a community chat function, which may include individual messaging, group messaging, and/or forums available to users of application 113. User interface element 147 may include a user interface element 147D, which may be interacted with by a patient to bring the patient to a doctor connection interface, which may include various mechanisms for communicating with the patient's doctor, including email, phone, chat, and/or video calling. User interface element 147 may include a user interface element 147E, which may be interacted with by a patient to bring the patient to a suggestion interface (shown in FIGS. 23A-23B). User interface element 147 may include a user interface element 147F, which may be interacted with by a patient to bring the patient to an interface to connect with a non-patient partner (e.g., an accountability partner).

FIGS. 26A-26B show exemplary user interfaces for activity partners and meetups, respectively. FIG. 26A shows a user interface 149 for connecting a patient with an activity partner (e.g., as described with respect to FIG. 24). A user may input a connection preference into user interface 149. User interface 149 may include a user interface element 149A, which may bring a patient to an activity partner interface and/or display a list of suitable activity partners. User interface 149 may include a user interface element 149B, which may bring a patient to a recovery mentee interface, where the patient may be presented with a list of potential mentors. User interface 149 may include a user interface element 149C, which may bring a patient to a recovery mentor interface, where the patient may be presented with a list of potential mentees (e.g., the reciprocal function of user interface element 149B).

FIG. 26B shows a user interface 151 of application 113 for connecting a patient with a meetup. User interface 151 may include a user interface element 151A, which may bring a patient to an active meetup interface. Application 113 may search for local meet ups with activity level or recovery period specified similar to the patient. User interface 151 may include a user interface element 151B which may bring a patient to a virtual meetup interface. Application 113 may search for virtual groups including patient members with activity level, activity interest or recovery period specified similar to the patient. User interface 151 may include a user interface element 151C which may bring a patient to a virtual meetup interface. Application 113 may search for local physical therapy group sessions grouped by members' reported pain levels, activity levels, and postoperative period timing.

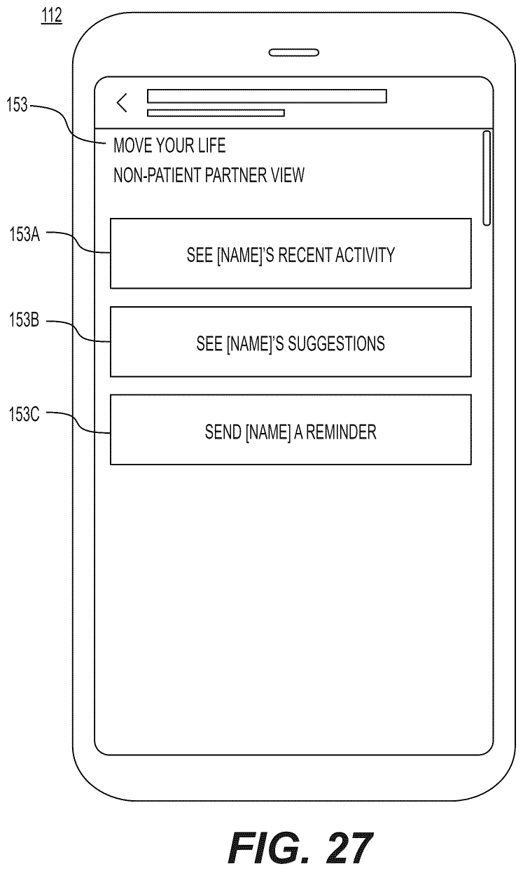

FIG. 27 shows a user interface 153 of application 113 for a non-patient partner. A non-patient partner a person may be a local person who serves as an accountability partner (e.g., friend, colleague, trainer, etc.) User interface 153 may include a user interface element 153A, which may bring a user (e.g., a non-patient partner) to a landing page with a matched patient's activity levels, may include preoperative activity levels 117 and/or postoperative activity levels 121. User interface 153 may include a user interface element 153B, which may bring a user to a landing page with a matched patient's suggestions (e.g., as shown in FIG. 23A-23B). This information may allow a non-patient partner to provide clinically relevant advice and input to a patient. User interface 153 may include a user interface element 153C, which may send a reminder to a matched patient (e.g., to perform physical activity). The reminder may be a predefined message or may be a custom message provided by the non-patient partner.

Aspects disclosed herein may be implemented during a robotic medical procedure where a robotic device, such as a surgical robot, a robotic tool manipulated or held by the surgeon and/or surgical robot, or other devices configured for automation perform at least a portion of a surgical procedure, such as a joint replacement procedure involving installation of an implant. Robotic device refers to surgical robot systems and/or robotic tool systems and is not limited to a mobile or movable surgical robot. For example, robotic device may refer to a handheld robotic cutting tool, jig, burr, etc.

Aspects disclosed herein are not limited to specific scores, thresholds, etc. that are described. For example, outputs and/or scores disclosed herein may include other types of scores such as Hoos Koos, SF-12, SF-36, Harris Hip Score, etc.

Aspects disclosed herein are not limited to specific types of surgeries and may be applied in the context of osteotomy procedures, computer navigated surgery, neurological surgery, spine surgery, otolaryngology surgery, orthopedic surgery, general surgery, urologic surgery, ophthalmologic surgery, obstetric and gynecologic surgery, plastic surgery, valve replacement surgery, endoscopic surgery, and/or laparoscopic surgery.

Aspects disclosed herein may improve or optimize surgical outcomes. Aspects disclosed herein may provide improved pain evaluation and patient connections to assist a patient's surgical recovery.

What is claimed is:

1. A method for providing relevant clinical connections, comprising:
receiving at least one of preoperative activity levels or postoperative activity levels associated with an instant patient from a patient database;
receiving a patient preference associated with the instant patient from the patient database;
determining, based on at least one of the preoperative activity levels or the postoperative activity, one or more potential connections from a plurality of users;
determining a time interval, wherein the time interval is based on a date of surgery for the instant patient and a present date;
determining a search area, wherein the search area is based on a current location of a user equipment associated with the instant patient;
determining one or more correlated users, based on (i) at least one of the preoperative activity levels or the postoperative activity levels and (ii) the one or more potential connections; and
displaying the one or more correlated users on an electronic display.

2. The method of claim 1, wherein the patient preference includes a travel preference and a connection preference, wherein the determining the search area is further based on the travel preference.

3. The method of claim 1, wherein the preoperative activity levels and the postoperative activity levels include kinematics data.

4. The method of claim 3, wherein the kinematics data is generated by a wearable sensor coupled to the instant patient, or a smart implant implanted in the instant patient.

5. The method of claim 4, wherein the kinematics data includes:
a range of motion, stiffness, or laxity of a joint, wherein the smart implant is installed at the joint.

6. The method of claim 1, wherein the connection preference includes at least one of an activity partner, a recovery mentor, or a recovery mentee, wherein the one or more potential connections are further based on a plurality of connections preferences, each of the plurality of connection preferences associated with a user of the plurality of users.

7. The method of claim 6, wherein each of the one or more potential connections have at least one characteristic in common with the instant patient.

8. The method of claim 7, wherein the one or more correlated users are displayed in descending order of most correlated with the instant patient to least correlated with the instant patient.

9. The method of claim 1, wherein the determining the one or more potential connections from the plurality of users is further based on pain data of the instant patient.

10. The method of claim 9, wherein the pain data of the instant patient is based on at least one of a facial expression classifier, a movement classifier, or a real-time sensor information classifier.

11. The method of claim 10, wherein the facial expression classifier is based on:
receiving visual media of the instant patient, the visual media including the instant patient performing at least one movement;
determining one or more parameters from the visual media using facial detection, facial alignment, and/or facial normalization;
determining at least one facial landmark or a facial texture from the determined one or more parameters; and
evaluating a facial pain instance based on the determined at least one facial landmark or facial texture.

12. The method of claim 11, wherein the at least one facial landmark includes eyes of the instant patient.

13. The method of claim 10, wherein the pain data is further based on a movement classifier, wherein the movement classifier is based on kinematics data generated by a wearable sensor coupled to the instant patient, or a smart implant implanted in the instant patient.

14. The method of claim 10, wherein the real-time sensor information classifier is based on at least one real-time measurement by a smart implant implanted in the instant patient.

15. The method of claim 3, wherein the preoperative activity levels and the postoperative activity levels further include range of motion data, alignment data, or joint stiffness data.

16. A method for providing relevant clinical suggestions, comprising:
receiving at least one of preoperative activity levels or postoperative activity levels associated with an instant patient from a patient database;
receiving pain data from a patient database, the pain data associated with the instant patient;
determining a time interval, wherein the time interval is based on a date of surgery for the instant patient and a present date;
determining, based on (i) the preoperative activity levels or the postoperative activity levels, (ii) the pain data, and (iii) the time interval, a clinical suggestion for the patient, wherein the clinical suggestion includes at least one physical activity.

17. The method of claim 16, wherein the pain data is based on a facial expression classifier, a movement classifier, and a real-time sensor information classifier.

18. The method of claim 17, wherein the facial expression classifier is based on:
receiving visual media of the instant patient, the visual media including the instant patient performing at least one movement;
determining at least one facial landmark or a facial texture using facial detection, alignment and/or normalization of the visual media; and
evaluating a facial pain instance based on the determined at least one facial landmark or facial texture.

19. A method for evaluating pain, comprising:
determining a facial expression classifier, including:
receiving visual media of an instant patient from a patient database, the visual media including the instant patient performing at least one movement;
determining at least one facial landmark or a facial texture using facial detection, alignment and/or normalization of the visual media; and
evaluating a facial pain instance based on the determining at least one facial landmark or facial texture;
determining a movement classifier based on kinematics data generated by a wearable sensor coupled to the instant patient, or a smart implant implanted in the instant patient;
determining a real-time sensor information classifier based on at least one real-time measurement by a smart implant implanted in the instant patient; and
displaying a pain metric associated with the instant patient, based on the facial expression classifier, the movement classifier, and the real-time sensor information classifier.

20. The method of claim 19, wherein the pain metric is a preoperative pain metric, the method further comprising:
comparing the preoperative pain metric to a postoperative pain metric; and
generating a clinical suggestion, based on the comparing the preoperative pain metric to the postoperative pain metric.
